(19) United States
(12) Patent Application Publication
(10) Pub. No.: US 2022/0275417 A1
(43) Pub. Date: Sep. 1, 2022

(54) METHOD FOR MANUFACTURING MONOCLONAL ANTIBODY USING YEAST, AND SCREENING METHOD

(71) Applicant: Barcodebody Inc., Kyoto-shi, Kyoto (JP)

(72) Inventor: Mitsuyoshi UEDA, Hyogo (JP)

(21) Appl. No.: 17/626,194

(22) PCT Filed: Nov. 8, 2019
(86) PCT No.: PCT/JP2019/044009

§ 371 Date: Jan. 11, 2022

(30) Foreign Application Priority Data
Jul. 22, 2019 (JP) 2019-134936

(51) Int. Cl. C12P 21/00; C12N 15/10; C12N 1/16; C12N 9/50; C12N 15/62; C12N 15/81; C07K 4/00; C07K 1/13

(52) U.S. Cl. CPC C12P 21/005; C12N 15/1065; C12N 1/165; C12N 9/50; C12N 15/625; C12N 15/815; C07K 4/00; C07K 1/13; C12N 2800/102; C12R 2001/84

(57) ABSTRACT

Disclosed is a method for manufacturing a monoclonal antibody without using animal individuals. This method includes a step of introducing a DNA fragment comprising a gene that encodes a secretory signal, a gene that encodes a nanobody, and a gene that encodes a peptide barcode, or a vector containing the DNA fragment, into a yeast cell; and a step of collecting a polypeptide comprising the nanobody and the peptide barcode that has been expressed in the cell and secreted to the outside of the cell. According to the method, it is possible to manufacture a monoclonal nanobody more efficiently in a shorter period of time without using animal individuals.

### TECHNICAL FIELD

The present invention relates to a method for manufacturing a monoclonal antibody using yeast, and a screening method.

### BACKGROUND ART

Antibodies are large protein molecules with a molecular weight of 150 kDa that each include heavy chains (H chains) and light chains (L chains). In a conventional method in which an antigen is administered to a vertebrate animal individual, for example, it takes time to produce antibodies. Paratopes of antibodies are encoded as genes at various sites of the genome. To cope with the entry of various foreign substances from the outside, various combinations of the paratopes are selected, and thus protein molecules that are so-called polyclonal antibodies are produced. Furthermore, monoclonal antibodies having improved specificity are manufactured by preparing hybridoma cells (fused cells) using myeloma cells in vitro and culturing the hybridoma cells.

As described above, polyclonal antibodies are manufactured through administration to vertebrate animals, and monoclonal antibodies are manufactured through in-vitro cell culture. However, full-body antibodies including heavy chains (H chains) and light chains (L chains) are large protein molecules, and therefore, both the method for manufacturing a polyclonal antibody and the method for manufacturing a monoclonal antibody are expensive and time-consuming methods.

On the other hand, when antibodies are manufactured, a display technique (e.g., phage display or yeast display) is used for screening of antibodies produced in animal bodies or cultured cells. With the display technique, antibodies are immobilized on the surface layer of a cell or phage. It is known that antibodies immobilized in this manner have improved structural stability. Accordingly, when antibodies selected through the screening process are used as free antibodies, which correspond to an actual usage form, the antibodies cannot be used due to loss of the structural stability in some cases. Moreover, a plurality of antibodies are presented to a single carrier (e.g., phage or yeast cell), and therefore, only antibodies having a low binding ability may be obtained due to the close inhibitory effect.

Monoclonal antibodies have gained attention as antibody drugs. A monoclonal antibody binds to a single antigen and has high specificity, and is thus expected to be applied to cancer cells, autoimmune diseases, infectious diseases, and the like. For example, a monoclonal antibody binds specifically to a target antigen, and thus the immune mechanism can attack and destroy cells containing the antigen (e.g., cancer cells or cells infected with an infectious factor). Accordingly, there is a demand for a method for more easily manufacturing a monoclonal antibody. Furthermore, there is also a demand for a method for more easily identifying a monoclonal antibody that binds specifically to a target antigen.

Non-Patent Documents 1 and 2 disclose that NestLink, which is a method for selecting and identifying a binding substance that can simultaneously characterize several thousand library members, was developed as a method for examining the binding ability of an antibody without immobilizing the antibody. NestLink is based on genetically encoded peptide barcodes “flycodes”, and Non-Patent Documents 1 and 2 disclose that these flycodes were designed to improve the detectability of mass spectrometry and serve as unique identifiers in sequencing analysis.

However, in order to more easily identify an antibody having a binding ability, there is still a demand for a method that has improved sensitivity and specificity, and reduced identification bias in peptide detection through mass spectrometry.

### RELATED ART DOCUMENTS

### Non-Patent Documents

[Non Patent Document 1] Egloff P. et al., bioRxiv: 287813 (Dec. 21, 2018)
[Non Patent Document 2] Egloff P. et al., NATURE METHOD VOL 16 May 2019 421-428

### SUMMARY OF THE INVENTION

### Problem to be Solved by the Invention

An object of the present invention is to provide a method for manufacturing a monoclonal antibody more efficiently in a shorter period of time.

### Means for Solving the Problem

The present invention provides a method for manufacturing a monoclonal antibody, comprising:

a step of introducing a DNA fragment comprising a gene that encodes a secretory signal, a gene that encodes a nanobody, and a gene that encodes a peptide barcode, or a vector containing the DNA fragment, into a yeast cell; and

a step of collecting a polypeptide comprising the nanobody and the peptide barcode that has been expressed in the cell and secreted to the outside of the cell.

In one embodiment, the yeast belongs to the genus Saccharomyces, Pichia, Schizosaccharomyces, Zygosaccharomyces, Candida, Torulopsis, Yarrowia, or Hansenula.

In one embodiment, the secretory signal is an α-factor secretory signal, a glucoamylase secretory signal, or a PHO1 secretory signal.

In one embodiment, the DNA fragment further comprises a promoter that is an AOX1 promoter, a GAP promoter, an FLD1 promoter, a PEX8 promoter, or a YPT1 promoter.

In one embodiment, the DNA fragment further comprises a gene encoding at least one tag selected from the group consisting of a FLAG tag, a His tag, a calmodulin protein (CBP) tag, a Strep tag, a StrepII tag, a GST tag, a Myc tag, and a maltose binding protein (MBP) tag.

In one embodiment, the peptide barcode is represented by an amino acid sequence having 6 to 16 amino acids, and the amino acids are independently selected from the group consisting of A, F, G, K, L, P, R, V, and W.

In one embodiment, the manufacturing method further comprises a step of mixing the collected polypeptide and an antigen and obtaining a polypeptide including a nanobody that binds specifically to the antigen;

a step of cleaving the peptide barcode from the obtained polypeptide and identifying the cleaved peptide barcode through mass spectrometry; and

a step of identifying the nanobody included in the polypeptide from which the identified peptide barcode was cleaved, based on the base sequence of a nucleic acid encoding the identified peptide barcode.

In one embodiment, the DNA fragment further comprises a specific protease cleavage site.

In one embodiment, the step of identifying the peptide barcode is performed by detecting a peak using a tandem mass spectrometer (MS/MS) connected to a high performance liquid chromatograph (LC).

In one embodiment, the high performance liquid chromatograph is provided with a long monolith column.

In one embodiment, the manufacturing method further comprises a step of determining the base sequence of the DNA fragment.

In one embodiment, at least two DNA fragments or vectors are used in the step of introduction into a yeast cell, and genes encoding a peptide barcode included in the DNA fragments encode peptide barcodes represented by different amino acid sequences.

In one embodiment, the DNA fragment includes a gene encoding two or more peptide barcodes, and a cleavage site is arranged at each position between the two or more peptide barcodes.

The present invention provides a vector for manufacturing a monoclonal antibody in yeast, wherein the vector contains a DNA fragment comprising a gene that encodes a secretory signal, a gene that encodes a nanobody, and a gene that encodes a peptide barcode, and is to be introduced into a cell of the yeast to express a polypeptide comprising the nanobody and the peptide barcode and secrete the polypeptide to the outside of the cell of the yeast.

In one embodiment, the secretory signal is an α-factor secretory signal, a glucoamylase secretory signal, or a PHO1 secretory signal.

In one embodiment, the DNA fragment further comprises a promoter that is an AOX1 promoter, a GAP promoter, an FLD1 promoter, a PEX8 promoter, or a YPT1 promoter.

In one embodiment, the DNA fragment further comprises a gene encoding at least one tag selected from the group consisting of a FLAG tag, a His tag, a calmodulin protein (CBP)tag, a Strep tag, a StrepII tag, a GST tag, a Myc tag, and a maltose binding protein (MBP) tag.

In one embodiment, the peptide barcode is represented by an amino acid sequence having 6 to 16 amino acids, and the amino acids are independently selected from the group consisting of A, F, G, K, L, P, R, V, and W.

In one embodiment, the DNA fragment further includes a specific protease cleavage site.

The present invention provides a screening method for a monoclonal antibody, comprising:

(i) a step of expressing an antibody library from a gene library,

the gene library comprising at least two gene members, each of the gene members comprising a DNA fragment that comprises a gene encoding a nanobody and a gene encoding at least one peptide barcode,

the DNA fragments of the gene members of the gene library encoding polypeptides of antibody members of the antibody library,

each of the polypeptides corresponding to the antibody members of the antibody library comprising a nanobody and at least one peptide barcode, the nanobody and the at least one peptide barcode being encoded by a DNA fragment comprised in a gene member of the gene library,

the peptide barcodes of the antibody members being represented by different amino acid sequences;

(ii) a step of mixing the antibody library and an antigen and selecting an antibody member of the antibody library that includes a nanobody binding to the antigen, from the antibody library;

(iii) a step of cleaving the peptide barcode included in the selected antibody member of the antibody library and identifying the cleaved peptide barcode through mass spectrometry; and

(iv) a step of determining the base sequence of the gene encoding the identified peptide barcode based on the base sequences of the gene library and identifying the nanobody of the antibody member from which the identified peptide barcode has been cleaved,

wherein the expression step is performed by introducing the above vector into a yeast cell.

In one embodiment, the screening method further comprising a step of determining the base sequence of the DNA fragment.

### Effects of the Invention

With the present invention, a monoclonal antibody that binds specifically to a desired antigen can be obtained in a short period of time. Moreover, with the present invention, the binding ability is measured using a free antibody, and therefore, a monoclonal antibody that binds specifically to an antigen can be obtained based on the binding ability of the antibody that corresponds to an actual usage form.

### BRIEF DESCRIPTION OF DRAWINGS

FIG. 1 is a schematic view showing an example of binding analysis based on antigen-antibody interaction of a nanobody with a peptide barcode manufactured through yeast secretory expression according to the present invention.

FIG. 2 shows schematic views of four types of nanobodies designed in Example 1.

FIG. 3 is an electrophoretic photograph showing the results of SDS-PAGE of anti-CD4-FLAG, anti-CD4-FLAG-barcode 1, anti-GFP-FLAG, and anti-GFP-FLAG-barcode 2 produced by Pichia pastoris transformants.

FIG. 4 is a graph showing the amounts of anti-CD4-FLAG, anti-CD4-FLAG-barcode 1, anti-GFP-FLAG, and anti-GFP-FLAG-barcode 2 produced by Pichia pastoris transformants.

FIG. 5 shows fluorescence micrographs showing the results of CD4 immunofluorescence staining using nanobodies with a peptide barcode.

FIG. 6 is a schematic view showing a scheme for quantifying the binding ability of nanobodies using mass spectrometry of peptide barcodes.

FIG. 7 shows graphs showing the LC-MS/MS analysis results of various amounts of the barcode 1 (A) and the barcode 2 (B).

FIG. 8 is a graph showing the results of LC-MS/MS quantifications of peptide barcodes cleaved from 250 fmol of the anti-CD4-FLAG-barcode 1 and 250 fmol of anti-GFP-FLAG-barcode 2.

FIG. 9 is a graph showing the results of LC-MS/MS quantifications of peptide barcodes cleaved from CD4-immobilized magnetic beads after 500 μL of a nanobody mixture (containing 0.1 μM anti-CD4-FLAG-barcode 1 and 0.1 μM anti-GFP-FLAG-barcode 2) was subjected to simultaneous binding assay using CD4-immobilized magnetic beads.

FIG. 10 is a graph showing peak capacities of monolith columns with inner diameters of 100 μm and 75 μm when these columns were used in an LC-MS/MS.

FIG. 11 is a graph showing peak capacities of monolith columns with lengths of 500 mm and 1000 mm when these columns were used in an LC-MS/MS.

### DESCRIPTION OF EMBODIMENTS

### Definitions

Essentially, terms that are commonly used in biology and immunology are used herein, but descriptions of the following terms will be given.

The term “nucleic acid” refers to a polymer form of nucleotides, deoxyribonucleotides, ribonucleotides, or analogues thereof that has any length. This term encompasses DNAs, RNAs, and modified forms thereof, for example. The nucleic acid may be linear or cyclic. The term “gene” refers to a region of a nucleic acid that contains genetic information encoded by a base sequence. A gene encodes a “peptide”, “polypeptide”, or “protein”, and a “peptide”, “polypeptide” or “protein” may be expressed based on the sequence information of the gene.

The term “peptide”, “polypeptide”, or “protein” refers to a polymer form of amino acids. A “peptide”, “polypeptide”, or “protein” includes, as constituent molecules, amino acids encoded by a nucleic acid, for example, and may also include, as constituent molecules, amino acids that are modified or derivatized chemically or biochemically. The terms “polypeptide” and “protein” are interchangeably used herein.

The term “antibody” as used herein means the same thing as what is commonly meant by the term that is used in biology and immunology, and refers to a polypeptide or protein that is also called an “immunogloblin”. A full-body antibody includes two heavy chains (H chains) and two light chains (L chains), and the chains are linked via disulfide bonds. The “antibody” as used herein includes any isotypic antibodies and antibody fragments that can bind specifically to an antigen, and examples thereof include Fab, Fv, scFv, Fd, V_{H}H (“nanobody”), chimeric antibodies, humanized antibodies, single-chain antibodies, and fusion proteins including an antigen binding portion of an antibody and a non-antibody protein. Antibodies can be detectably labeled with a radioisotope, an enzyme that produces a detectable product, a fluorescent protein, a peptide barcode and the like.

A “nanobody”, which is also called a single-domain antibody, is one type of antibody fragment that is constituted by a single monomeric variable antibody domain and does not include light chains and the CH domains of heavy chains in the conventional Fab region. Nanobodies have a molecular weight of 12 to 15 kDa, which is about a tenth of the molecular weight (150 kDa) of common full-body antibodies. Despite such a small molecular weight, nanobodies have the same characteristics as those of full-body antibodies. Examples of nanobodies include antibodies constituted by a V_{H}H domain, which is a variable region of a single-domain camelid antibody. Commonly, nanobodies are polypeptides including about 120 amino acid residues. Basically, nanobodies have a sequence configuration similar to that of a variable region of a typical immunogloblin, and in this configuration, three hypervariable regions, which are also called complementarity-determining regions (CDRs), namely CDR1, CDR2, and CDR3, are located with four framework regions, namely FR1, FR2, FR3, and FR4, being located therebetween.

A “gene library” is a group including at least two genes, and genes included in the group are also referred to as “gene members”. In the present invention, genes serving as the gene members of the gene library group can be inserted into separate vectors (e.g., plasmids) and thus be independently present. Furthermore, these genes or vectors may be introduced into separate host cells (e.g., yeast cells) and thus be individually present. An “antibody library” is a group including at least two antibodies, and antibodies included in the group are also referred to as “antibody members”. In the present invention, the antibody members of the antibody library are obtained through expression of the gene members of the gene library in the host cells. The antibody members of the antibody library group can be present as free antibodies in the supernatant of the culture of the host cells used to express the gene members ofthe gene library, for example.

The term “base sequence” refers to an alignment order of continuous nucleotide bases in a gene, and abase sequence carries genetic information. The term “amino acid sequence” refers to an alignment order of continuous amino acids in a peptide or protein.

A gene “encoding” a peptide or protein is transcribed (in the case of a DNA) and translated into the peptide or protein (in the case of an mRNA) when the gene is brought under the control of an appropriate control element or regulatory element, for example.

The “peptide barcode” refers to a peptide having a sequence that can be used to identify a molecule (e.g., antibody) to which a peptide barcode is fused, and/or distinguish such a molecule from one or more different molecules. It is preferable that the peptide barcode is fused to an antibody so as not to have an influence on binding, to an antigen, of the antibody to which the peptide barcode is fused, has such a size that does not have such an influence thereon, and has such an amino acid sequence that does not have such an influence thereon. In this specification, “fusing” of a peptide (e.g., peptide barcode) to a molecule (e.g., antibody or tag) means that the molecule is linked to the C-terminus or N-terminus of the peptide to form a larger molecule, but another molecule or region (e.g., cleavage site) may be arranged between the peptide and the molecule, and/or one or more (e.g., two to dozens) amino acids may be present between the peptide and the molecule. “Peptide barcodes” are encoded by genes, and the base sequences of the gene encoding peptide barcodes can serve as gene barcodes.

“Specific binding” as used herein refers to binding based on interaction between binding partners (e.g., antigen and antibody) that bind to each other but do not sufficiently or substantially bind to the other molecules that may be present in the environment (e.g., biological sample or tissue), under given conditions (e.g., physiological conditions).

(1. Method for Manufacturing Monoclonal Antibody)

The present invention provides a method for manufacturing a monoclonal antibody. This manufacturing method includes: a step of introducing a DNA fragment including a gene that encodes a secretory signal, a gene that encodes a nanobody, and a gene that encodes a peptide barcode, or a vector containing the DNA fragment, into a yeast cell (step (A)); and a step of collecting a polypeptide including the nanobody and the peptide barcode that has been expressed in the cell and secreted to the outside of the cell (step (B)).

### (1-1. Step (A): Yeast Secretory Expression)

In the present invention, a monoclonal antibody is manufactured through yeast secretory expression. Yeast does not contain endotoxin, and facilitates a process for purifying an antibody obtained through expression. Examples of yeast include yeast belonging to the genera Saccharomyces, Pichia, Schizosaccharomyces, Zygosaccharomyces, Candida, Tonulopsis, Yarrowia, and Hansenula. Examples thereof include Saccharomyces cerevisiae, Schizosaccharomyces pombe, and Pichia pastons. Pichia pastoris is preferable.

Any secretory signals can be used as long as host yeast in which a monoclonal antibody is expressed can secrete the monoclonal yeast to the outside of the cell. From the viewpoint of efficient yeast secretory expression, an α-factor secretory signal (e.g., α-factor prepro sequence derived from Saccharomyces cerevisiae), a glucoamylase secretory signal (e.g., secretory signal for glucoamylase derived from Rhizopus oryzae), or a PHO1 secretory signal (e.g., secretory signal for phosphatase (PHO1) derived from Pichia pastoris) is used.

In the present invention, a monoclonal antibody can be manufactured through expression of the gene encoding a nanobody. The nanobody is constituted by a single variable domain (VHH) of an antibody heavy chain, and this variable domain can bind to only one type of antigen. The gene encoding a nanobody may have a known base sequence or an unknown base sequence. A chimeric nanobody may also be employed.

When the base sequence information is known, the gene encoding a nanobody can be obtained through PCR using, as a template, an available genome DNA, cDNA, or the like with a primer pair designed based on the sequence information. An artificial gene may be produced by chemically synthesizing a DNA having the base sequence. The gene encoding a nanobody can also be obtained by amplifying, through PCR or the like, a variable region derived from a DNA extracted from a leukocyte in the thymus, bone marrow, peripheral blood, or the like of a vertebrate including a camelid, for example.

Various mutations may be introduced into the gene encoding a nanobody through exhaustive mutagenesis using NNK codons, for example, and error-prone PCR can be used together. With these techniques, genes encoding various antibodies may be designed without using animal individuals.

The above-mentioned DNA fragment can further include a promoter. The promoter can be arranged upstream of the secretory signal. Any promoters that can lead gene expression in yeast can be used. The above-mentioned DNA fragment can further include a promoter. The promoter may be an inducible promoter or a constitutive expression promoter. Examples of the inducible promoter include an AOX1 promoter (promoter for the gene encoding alcohol oxidase (AOX)), an FLD1 promoter (promoter for the gene encoding formaldehyde dehydrogenase (FLD)), and a PEX8 promoter (promoter for the gene encoding one of peroxins (PEXs)), and examples of the constitutive expression promoter include a GAP promoter (promoter for the gene encoding glyceraldehyde-3-phosphate dehydrogenase) and a YPT1 promoter (these promoters may be derived from Pichia pastoris). When the yeast is Pichia pastoris, a methanol inducible promoter (e.g., AOX1 promoter, FLD1 promoter, or PEX8 promoter (mentioned above)) can be used, and a constitutive expression promoter may also be used. Methanol inducible promoters are favorably used in methanol-utilizing yeast.

The above-mentioned DNA fragment may further include a gene encoding a protein tag. Examples of the protein tag include a FLAG tag, a His tag, a CBP (calmodulin protein) tag, a Strep tag, a StrepII tag, a GST tag, a Myc tag, an MBP (maltose binding protein) tag, and combinations thereof. Addition of such a tag makes it easier to collect and purify a monoclonal antibody secreted to the outside of a yeast cell and makes it possible to perform mass spectrometry (MS) of a peptide barcode with higher precision.

The base sequence of the gene encoding a peptide barcode can be produced based on the amino acid sequence of a peptide barcode, which will be described below. The amino acid sequence of the peptide barcode includes, for example, 5 to 30 amino acids, preferably 6 to 20 amino acids, more preferably 6 to 16 amino acids, and even more preferably 6, 7, 8, 9, or 10 amino acids. Due to the peptide barcode having an amino acid sequence with a length within this range, the ionization efficiency of the peptide barcode is improved, and therefore, the peptide barcode can be more favorably detected and quantified through mass spectrometry and can be easily identified. When the length of the amino acid sequence is within the range mentioned above, it is possible to verify in advance that a designed peptide barcode has a high ionization efficiency, and identification through mass spectrometry (MS) is less likely to be biased. Therefore, the binding ability of all candidate nanobodies can be measured without omission. For example, when peptide barcodes including 6, 7, 8, 9, or 10 amino acids are employed, the detection sensitivity in mass spectrometry is improved, the antigen binding ability of a nanobody with a barcode is not substantially impaired, and various barcodes can be produced. Alternatively, when such peptide barcodes are employed, it is also possible to further add a protein tag to the peptide barcode without substantial influence on the binding ability of a nanobody and the detection sensitivity in mass spectrometry.

In one embodiment, amino acids constituting a peptide barcode are independently selected from the group consisting of alanine (A), phenylalanine (F), glycine (G), lysine (K), leucine (L), proline (P), arginine (R), valine (V), and tryptophan (W). Regarding the ionization efficiency, ion suppression does not occur in these amino acids. For example, residues having an amino group, such as lysine (K) and arginine (R), are likely to be positively charged, and improve the ionization efficiency. A glycine (G) residue and a proline (P) residue also improve the ionization efficiency. On the other hand, histidine (H) reduces the ionization efficiency, cysteine (C) and methionine (M) are likely to be oxidized, and asparagine (N), serine (S), and threonine (T) can be glycosylated. Therefore, these amino acids may be excluded from amino acids to be included in peptide barcodes. Furthermore, hydrophobic amino acids are also included in order to adequately vary the elution time (retention time) in a liquid chromatograph (LC). In one embodiment, a peptide barcode in which A, F, G, K, L, P, R, V, and W are randomly arranged is used. Employing these types of amino acids to form peptide barcodes makes it possible to produce peptide barcodes having a high ionization efficiency using the above-mentioned number of amino acids (particularly 6, 7, 8, 9, or 10 amino acids). Accordingly, identification through mass spectrometry (MS) is less likely to be biased, and the binding ability of all candidate nanobodies can be measured without omission. It is also possible to form various barcodes which can also moderately vary the retention time in separation using a liquid chromatograph (LC) that is performed prior to mass spectrometry (MS) to be used to identify peptide barcodes and thus improve the specificity of detection by mass spectrometry.

For example, when peptide barcodes including 6 amino acids are produced using the types of amino acids as mentioned above, the number of unique barcodes that can be designed is 9⁶ (=531,441).

The DNA fragment can include a gene encoding a cleavage site, which can be degraded by an enzyme or chemical means. As described below, in order to make it easier to cleave a peptide barcode, it is preferable that the cleavage site can be cleaved by a protease that has cleavage specificity. It is preferable to provide an amino acid sequence recognized by a protease having cleavage specificity (also referred to as a “specific protease” herein). An example of the specific protease is enterokinase, but is not limited thereto. For example, when the specific protease is enterokinase, the downstream side of lysine in an amino acid sequence DDDDK (four aspartic acids and lysine) (i.e., the C-terminal side of a peptide including the above-mentioned 5 amino acids) is cleaved.

Codons optimized depending on a host may be employed in the base sequences of the genes.

The DNA fragment can further include a terminator. Any terminators can be used as long as they function in yeast. An example thereof is an AOX1 terminator.

Synthesis and coupling of DNAs including various constituent elements (i.e., various genes, a promoter, and a terminator) can be performed using techniques that are commonly used by a person skilled in the art. For example, a gene encoding a secretory signal peptide and a gene encoding a nanobody can be coupled using site-directed mutagenesis. Using this technique enables more correct cleavage of the secretory signal peptide and expression of the nanobody.

The DNA fragment constructed as described above is introduced into a yeast cell. The DNA fragment may be inserted into a vector and then introduced thereinto. As the constituent elements of the DNA fragment (e.g., genes encoding a promoter and a terminator, and/or a secretory signal), those provided in a commercially available yeast expression vector in advance may be used. “Introducing” means not only introducing the genes in the DNA fragment into a cell but also expressing the genes therein. Examples of such an introduction technique include transformation, transduction, transfection, co-transfection, and electroporation. In the case of introduction into a yeast cell, specific examples thereof include a technique using lithium acetate and a protoplast technique. The introduced DNA fragment may be incorporated into a chromosome through insertion into a host gene or homologous recombination with a host gene. A commercially available kit can also be used for introduction. In the case where the host cell is a yeast cell, Frozen EZ Yeast Transformation II Kit (manufactured by Zymo Research) is used, for example.

### (1-2. Step (B): Collection of Polypeptide Produced Through Secretory Expression)

Due to the introduction as mentioned above, a polypeptide including the nanobody and the peptide barcode is expressed in the yeast cell and then secreted to the outside of the cell. For example, in the case where Pichia pastoris, which is methanol-utilizing yeast, is used as a host cell, a plasmid vector including a DNA fragment in which, for example, a gene encoding a fusion protein of a secretory signal peptide (e.g., α-factor secretory signal peptide) and a nanobody with a peptide barcode is arranged downstream of a methanol inducible promoter (e.g., AOX1 promoter) is designed. When such a plasmid vector is introduced into a host cell to express a fusion protein, the α-factor secretory signal is cleaved during the secretory process, and the nanobody with a peptide barcode is finally secreted into the culture medium supernatant. By collecting the culture supernatant, the polypeptide including the nanobody and the peptide barcode can be collected. Without the need to disrupt the cell to obtain the expressed antibody, the polypeptide including the nanobody and the peptide barcode can be collected. The polypeptide (monoclonal antibody with a peptide barcode) may be purified from the culture supernatant as needed.

The manufacturing method of the present invention can further include: a step of mixing the collected polypeptide and an antigen and obtaining a polypeptide including a nanobody that binds specifically to the antigen (step (C)); a step of cleaving the peptide barcode from the obtained polypeptide and identifying the cleaved peptide barcode through mass spectrometry (step (D)); and a step of identifying the nanobody included in the polypeptide from which the identified peptide barcode was cleaved based on the base sequence of a nucleic acid encoding the identified peptide barcode (step (E)).

(1-3. Step (C): Binding Based on Antibody-Antigen Interaction)

In order to more reliably obtain a monoclonal antibody that binds specifically to a target antigen, the polypeptide collected in step (B) may be mixed with the antigen, and a polypeptide including a nanobody that binds specifically to the antigen may be obtained (step (C)). When the culture supernatant is collected, for example, in accordance with step (B) above, this culture supernatant contains a nanobody with a peptide barcode and can thus be used in binding assay for examining the antigen binding ability as it is. Without the need to disrupt the cell to obtain the expressed antibody, the culture supernatant can be used to measure the binding ability of an antibody as it is, thus making it possible to measure the antigen binding ability in a short period of time at low cost without the need for pre-purification.

There is no limitation on the antigen as long as it can induce an immune reaction to produce an antibody, and examples thereof include proteins, peptides, polysaccharides, lipids, low-molecular-weight compounds, and cells collected from living organisms. Proteins are preferable.

The antigen can be immobilized on a carrier. Magnetic beads (e.g., NHS-activated magnetic beads (manufactured by Thermo Scientific)) can be used as such a carrier. For example, a cell (e.g., yeast cell) that displays the antigen on its surface layer can also be used. Moreover, a cell sorter (FCS) can also be utilized. Such binding assay for antigen-antibody binding can be performed in accordance with the techniques and conditions that are commonly used by a person skilled in the art. For example, when magnetic beads on which the antigen is immobilized are used, a reaction for binding the antigen and an antibody is caused, and then the beads are washed with an appropriate solvent to wash away polypeptides including an antibody that does not bind to the antigen. Thereby, a polypeptide including an antibody that binds to the antigen can be retained on the beads.

Assay based on surface plasmon resonance can also be used, for example, in order to examine the antigen binding ability. Such assay can be performed by measuring kinetic parameters using Biacore T-200 (manufactured by GE Healthcare), for example.

(1-4. Step (D): Identification of Peptide Barcode)

In order to identify and/or obtain the antibody that is found to bind to the antigen, it is possible to cleave the peptide barcode from the polypeptide in which the nanobody has bound to the antigen in step (C) and identify the cleaved peptide barcode through mass spectrometry (step (D)).

From the polypeptide in which the nanobody has bound to the antigen, the peptide barcode, which is included in the polypeptide together with the nanobody, is cleaved. Such a peptide barcode is cleaved by adding an enzyme that recognizes a cleavage site to cleave the polypeptide at the cleavage site, for example. It is preferable to use a specific protease (e.g., enterokinase). Thus, the antibody that binds to the antigen is retained, and the peptide barcode is removed. This removed peptide barcode can be collected.

Then, the cleaved peptide barcode is identified through mass spectrometry. For example, the peptide barcode can be identified by comparing the molecular weight determined based on a peak detected through mass spectrometry with the molecular weight estimated from the configuration (amino acid sequence) of the peptide barcode.

For example, a mass spectrometer (MS) or tandem mass spectrometer (MS/MS) that is connected to a high performance liquid chromatograph (LC) can be used to perform such mass spectrometry (i.e., LC-MS technique or LC-MS/MS technique). The LC-MS/MS technique is preferable from the viewpoint that the specificity of detection is improved. A triple quadrupole mass spectrometer (e.g., LCMS-8060: manufactured by Shimadzu Corporation) is preferable as the tandem mass spectrometer from the viewpoint of high sensitivity, specificity, and quantitativeness. Selected reaction monitoring (SRM: a technique of detecting a product ion having specific m/z generated through dissociation of a precursor ion having specific m/z) is preferably used as a measurement technique for MS/MS.

For example, a monolith column, preferably a long monolith column, is used as a column for the liquid chromatograph (LC). The monolith column is a rod-shaped integral-type liquid chromatography column provided with two types of pores that are different in size (mesopores and macropores), and monolith silica gel is used as a separation medium. The long monolith column refers to a monolith column having a column length of 100 mm to 10000 mm, for example. The monolith column has an inner diameter of 1 μm to 200 μm, and preferably 30 μm to 75 μm, for example. It is more preferable to use a long monolith column having an inner diameter within the range as mentioned above because the column separation capacity is improved, and a peptide barcode can thus be more efficiently detected and more easily identified. Also, using a long monolith column makes it possible to perform absolute quantification of a peptide through SRM.

(1-5. Step (E): Identification of Monoclonal Antibody)

Based on the base sequence of the gene encoding the peptide barcode identified as described above, it is possible to identify an antibody included in the polypeptide including the identified peptide barcode (step (E)).

When the peptide barcode is identified through mass spectrometry, the nanobody with the peptide barcode can be estimated from the base sequence of the gene encoding the peptide barcode. It is possible to obtain the base sequence information of the nanobody estimated as described above to identify the nanobody. For example, when a gene encoding a nanobody has an unknown base sequence, the manufacturing method of the present invention may further include a step of determining the base sequence of the above-mentioned DNA fragment in order to obtain the base sequence of the gene encoding a nanobody. A technique that is commonly used by a person skilled in the art can be used to determine the base sequence as described above.

In this manner, a monoclonal antibody that binds specifically to a target antigen can be identified.

Furthermore, the identified monoclonal antibody can also be obtained through expression of the gene encoding the antibody. For example, a primer pair is designed based on the base sequence information of the nucleic acid encoding the identified antibody, and the nucleic acid encoding the antibody is obtained through PCR using the primer pair. An expression vector is constructed using such a nucleic acid as mentioned above, and this expression vector is introduced into a host cell, thus making it possible to obtain an expressed antibody. It is preferable to use the yeast secretory expression as described above for such expression of a monoclonal antibody.

Two or more DNA fragments can also be used in the manufacturing method of the present invention. In this case, at least two DNA fragments or vectors are used in the step of introduction into a yeast cell (step (A)), and genes encoding a peptide barcode included in the DNA fragments encode peptide barcodes represented by different amino acid sequences. When two or more DNA fragments are used, designing the DNA fragments such that peptide barcodes have different amino acid sequences makes it possible to identify the peptide barcodes detected through mass spectrometry in step (D).

The at least two DNA fragments or vectors can be separately introduced into a corresponding number of yeast cells. A plurality of DNA fragments or vectors may also be introduced into one yeast cell. The number of the at least two DNA fragments or vectors need only be at least two or more, and can be set to 2 to 10⁸, and preferably 10² to 10⁷ or 10³ to 10⁶, for example).

Moreover, one DNA fragment may also include a gene encoding two or more peptide barcodes. In this case, the two or more peptide barcodes can be arranged in series for a nanobody encoded by one DNA fragment. A cleavage site can be arranged at each position between the two or more peptide barcodes. For example, the variety of peptide barcodes can be increased by arranging two or more peptide barcodes in series and arranging a specific protease cleavage sequence at each position between the peptide barcodes. For example, when a library is constructed using polypeptides having a sequence NH₂-[nanobody]-cleavage site-[peptide barcode A]-cleavage site-[peptide barcode B]-COOH, where a peptide barcode close to the COOH-terminus is named “peptide barcode B” and a peptide barcode close to the NH₂-terminus is named “peptide barcode A”, one amino acid serving as an identifier is added to a terminus (which may be the C-terminus or N-terminus) of each peptide barcode such that the amino acids vary depending on peptide barcodes and the positions of the peptide barcodes can be identified. Any of A, F, G, K, L, P, R, V, and W can be used as the amino acid serving as an identifier. For example, when K (lysine) is added to the C-terminus of the peptide barcode A and R (arginine) is added to the C-terminus of the peptide barcode B, it is possible to distinguish a peptide barcode derived from the peptide barcode A and a peptide barcode derived from the peptide barcode B through mass spectrometry. In this case, when two peptide barcodes are arranged in series, evaluation can be performed using a wide variety of types of combinations (9⁶)²=531,441²). When three types of peptide barcodes are arranged in series, evaluation can be performed using a wider variety of types of combinations (9⁶)³=531,441³). Four or more peptide barcodes can be arranged in series, and thus the quantifiable nanobody library increases in size. One DNA fragment includes two to five peptide barcodes, for example.

(2. Vector for Manufacturing Monoclonal Antibody in Yeast)

The present invention further provides a vector for manufacturing a monoclonal antibody in yeast. The vector of the present invention contains the above-mentioned DNA fragment. The constituent elements (a gene encoding a secretory signal, a gene encoding a nanobody, and a gene encoding a peptide barcode, and a promoter, a gene encoding a protein tag, a gene encoding a cleavage site, and a terminator, as described above) of the DNA fragment are as described above.

The vector can include factors such as a selective marker, a replication origin, and an enhancer as appropriate. The vector is in a plasmid form, for example. For example, a plasmid including a yeast ColE1 replication origin is favorably used. It is preferable that the plasmid includes a selective marker from the viewpoint of facilitating the plasmid preparation and the detection of a transformant. Examples of the selective marker include drug resistance genes and auxotrophic genes. Examples of the drug resistance genes include, but are not particularly limited to, an ampicillin resistance gene (Ampr) and a kanamycin resistance gene (Kanr). Examples of the auxotrophic genes include, but are not particularly limited to, an N-(5′-phosphoribosyl)anthranilate isomerase (TRP1) gene, a tryptophan synthase (TRP5) gene, a β-isopropylmalate dehydrogenase (LEU2) gene, an imidazoleglycerol-phosphate dehydrogenase (HIS3) gene, a histidinol dehydrogenase (HIS4) gene, a dihydroorotate dehydrogenase (URA1) gene, and an orotidine-5-phosphate decarboxylase (URA3) gene. A replication gene for yeast can be selected as needed.

(3. Screening Method for Monoclonal Antibody)

The present invention also provides a screening method for a monoclonal antibody. This method includes:

(i) a step of expressing an antibody library from a gene library,

the gene library including at least two gene members, each of the gene members including a DNA fragment that includes a gene encoding a nanobody and a gene encoding at least one peptide barcode,

the DNA fragments included in the gene members of the gene library encoding polypeptides corresponding to antibody members of the antibody library,

each of the polypeptides corresponding to the antibody members of the antibody library including a nanobody and at least one peptide barcode, the nanobody and the at least one peptide barcode being encoded by a DNA fragment included in a gene member of the gene library,

the peptide barcodes of the antibody members being represented by different amino acid sequences;

(ii) a step of mixing the antibody library and an antigen and selecting an antibody member of the antibody library that includes a nanobody binding to the antigen, from the antibody library;

(iii) a step of cleaving the peptide barcode included in the selected antibody member of the antibody library and identifying the cleaved peptide barcode through mass spectrometry; and

(iv) a step of determining the base sequence of the gene encoding the identified peptide barcode based on the base sequences of the gene library and identifying a nanobody included in the antibody member from which the identified peptide barcode has been cleaved.

The expression step (step (i)) mentioned above is performed by introducing a vector as described in “2. Vector for Manufacturing Monoclonal Antibody in Yeast” above into a yeast cell.

(3-1. Step (i): Expression of Gene Library)

With the present invention, by expressing a plurality of types of nanobodies simultaneously, a monoclonal antibody that binds specifically to a target antigen can also be selected and identified. A gene library can be produced for this purpose. The gene library includes at least two gene members, and each of the gene members includes a DNA fragment that includes a gene encoding a nanobody and a gene encoding at least one peptide barcode. This DNA fragment can be constructed as described above and inserted into a vector as needed.

The DNA fragments in the gene members of the gene library encode polypeptides corresponding to antibody members of an antibody library. Accordingly, each of the polypeptides corresponding to the antibody members of the antibody library includes a nanobody and at least one peptide barcode. The nanobody and the at least one peptide barcode of each antibody member are encoded by the DNA fragment included in the gene member of the gene library.

The peptide barcodes of the antibody members of the antibody library are represented by different amino acid sequences. The antibody members of the antibody library are randomized antibodies with a unique peptide barcode, and the gene members of the gene library are randomized antibody genes (nucleic acids encoding antibodies) with a gene (nucleic acid (e.g., DNA)) encoding a unique peptide barcode (i.e., unique DNA barcode). Accordingly, the randomized antibody genes and the DNA barcodes have a one-on-one relationship, and the randomized antibodies and the peptide barcodes have a one-on-one relationship. The peptide barcodes of the antibody members of the antibody library can be efficiently detected through mass spectrometry.

Since the peptide barcodes of the antibody members have different amino acid sequences, the nucleic acids (DNA barcodes) encoding the peptide barcodes have different base sequences. The amino acid sequences of the peptide barcodes of the antibody members can be designed such that different retention times are obtained in a liquid chromatograph (LC)-tandem mass spectrometry (MS/MS), which can be used for the identification by mass spectrometry, when they are separated with an LC, and the base sequences of the genes can be designed such that the genes encode the designed amino acid sequences.

Accordingly, when the base sequence information of a DNA barcode can be obtained, the base sequence of the antibody gene (nucleic acid encoding an antibody) with the DNA barcode can be determined. In the nucleic acid library, the nucleic acids encoding a peptide barcode in the nucleic acid members are different, but some of the nucleic acid members may encode the same antibody (in other words, one or more types (e.g., 2 to 10 types) of peptide barcodes may be added to one type of antibody). This makes it possible to provide for a case where a certain nucleic acid member fails to be expressed or a case where an antibody with a peptide barcode is lost prior to mass spectrometry for some reason.

In one embodiment, an antibody member of the antibody library includes two or more peptide barcodes, and a cleavage site is arranged at each position between the two or more peptide barcodes. The peptide barcodes and the cleavage site are as described above. Arranging two or more peptide barcodes in series makes it possible to increase the number of barcodes and to achieve the variety that is greater than or equal to that observed in mammals, for example.

The number of the gene members included in the gene library need only be at least two or more, and can be set to 2 to 10⁸, and preferably 10² to 10⁷ or 10³ to 10⁶, for example. When the number of the gene members is within these ranges, it is possible to more efficiently obtain the base sequences of the gene library and/or perform simultaneous binding assay based on antigen specific binding.

A gene library may be newly constructed by designing sequences for screening of a monoclonal antibody that binds specifically to an antigen, or a gene library prepared in advance using the existing sequence information may be used.

The antibody library is expressed from the gene library in the same manner as in the above description of the method for manufacturing a monoclonal antibody and the vectors. That is, DNA fragments corresponding to the gene members of the gene library are introduced into yeast cells, and nanobodies and peptide barcodes are produced through yeast secretory expression. The above-described vectors are used for this yeast secretory expression.

(3-2. Step (ii): Selection of Antigen Binding Antibody Member)

In the screening method of the present invention, the antibody library and an antigen are mixed, and an antibody member of the antibody library including an antibody that binds to the antigen is selected from the antibody library (step (ii)). In this step, the antibody library is subjected to screening for the purpose of examination of binding to a target antigen. After the introduction into a yeast cell in step (i) above, the culture medium supernatant contains the antibody members (nanobodies with a peptide barcode) of the antibody library due to yeast secretory expression. Accordingly, the culture medium supernatant may be added to an antigen as it is in order to mix the antibody library and the antigen. Polypeptides may be purified from the culture medium supernatant as needed and then added to an antigen. This step (ii) can be performed basically in the same manner as “Step (C): Binding Based on Antibody-Antigen Interaction” above (step (C)). It is preferable to use simultaneous binding assay in which the antibody members of the antibody library are simultaneously added to an antigen.

(3-3. Step (iii): Identification of Peptide Barcode)

In the screening method of the present invention, a peptide barcode included in the selected antibody member of the antibody library is cleaved, and the cleaved peptide barcode is identified through mass spectrometry (step (iii)). This step (iii) can be performed basically in the same manner as “Step (D): Identification of Peptide Barcode” above.

(3-4: Step (iv): Identification of Monoclonal Antibody)

In the screening method of the present invention, the base sequence of a gene encoding the identified peptide barcode is determined based on the base sequences of the gene library, and a nanobody included in the antibody member from which the identified peptide barcode has been cleaved is identified (step (iv)). This step (iv) can be performed basically in the same manner as “Step (E): Identification of Monoclonal Antibody” above.

In a case where a nucleic acid having an unknown base sequence is included or a case where the combination of a peptide barcode and an antibody is unknown, a step of obtaining the base sequences of the gene library can be further included.

The base sequences of the gene library can be obtained by performing large-scale sequencing analysis on the gene library. Large-scale sequencing analysis can be performed in accordance with a technique that is commonly used by a person skilled in the art, and can be performed using a DNA sequencer that can process a large quantity of base sequence information, for example. A next-generation sequencer (NGS) such as MiSeq (manufactured by illumina K.K.) can be used for large-scale sequencing analysis.

A database may also be produced using the base sequence information. After a peptide barcode is identified through mass spectrometry, such a database can be used to identify an antibody based on the base sequence information of a nucleic acid (including a gene that encodes a peptide barcode and a gene that encodes an antibody) encoding the member of the antibody library including the identified peptide barcode, and the sequence information of the amino acid sequence (including a peptide barcode and an antibody) estimated based on the base sequence information as needed.

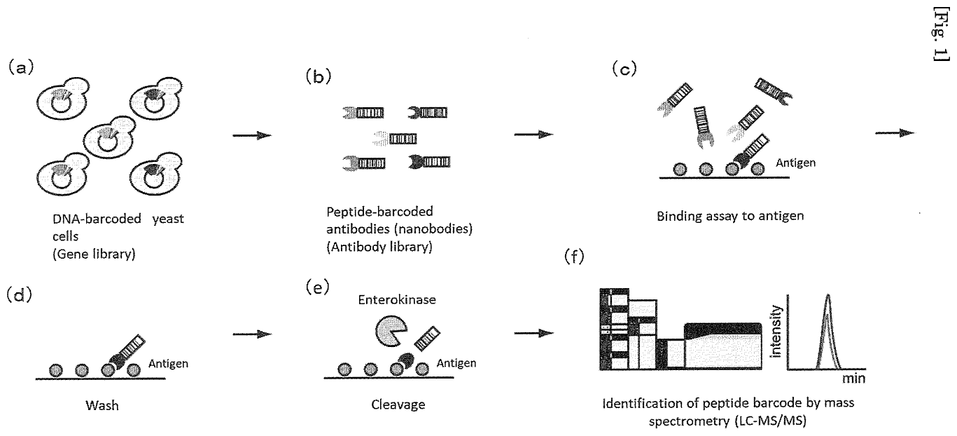

The present invention will be further described with reference to FIG. 1.

FIG. 1 is a schematic view showing an example of binding analysis based on antigen-antibody interaction of a nanobody with a peptide barcode manufactured through yeast secretory expression according to the present invention. In FIG. 1, (a) a gene library including at least two gene members that are randomized nanobody genes (genes encoding a nanobody) with a unique DNA barcode (a gene encoding a peptide barcode) is provided, and the gene members are introduced into yeast cells; (b) antibody members including antibodies (nanobodies) with a peptide barcode are produced through expression in the yeast cells to form an antibody library, and the antibody members of this antibody library is secreted into the culture medium supernatant from the yeast cells; (c) the antibody members of this antibody library is simultaneously mixed with an antigen and allowed to bind to the antigen; (d) antibody members including a non-binding antibody are removed through washing, and an antibody member including an antibody binding to the antigen is retained and selected; (e) the peptide barcode is cleaved from the selected antibody member using enterokinase; and (f) the cleaved peptide barcode is identified through mass spectrometry.

The one-on-one correspondence relationship between the base sequences of the randomized antibody genes and the base sequences of the DNA barcodes (genes encoding a peptide barcode) can be determined based on the sequence information of the gene library in FIG. 1(a), and the correspondence relationship between the amino acid sequences (encoded by the base sequences of the antibody genes) of the randomized antibodies and the amino acid sequences of the peptide barcodes can be deductively examined. When an antibody member including the antibody binding to an antigen is selected from the antibody library, a peptide barcode is cleaved from the selected antibody member, and the selected peptide barcode is identified through mass spectrometry, as shown in (b) to (f) mentioned above, it is possible to determine the base sequence of the gene encoding the antibody member including the identified peptide barcode using the known sequence information of the nucleic acid library or the sequence information obtained through sequencing analysis together, and to determine which antibody binds specifically to the antigen, thus making it possible to obtain a monoclonal nanobody against the antigen. Furthermore, the thus-identified antibody can also be manufactured by obtaining a gene encoding the antibody based on the sequence information of the nucleic acid library and expressing the antibody from the gene.

As described above, with the present invention, a monoclonal antibody can be produced in vitro without using animals, and, through mass spectrometry of a unique peptide barcode added to a free antibody, the binding ability of the free antibody can be identified. Moreover, with the present invention, antibodies having various properties can be produced in vitro or in a laboratory instead of producing antibodies using expensive and time-consuming methods in which vertebrates or cultured cells are used. Furthermore, with the present invention, when many nanobodies serving as candidates for monoclonal antibodies are used, the ability of these candidate nanobodies to a target antigen can be comprehensively identified in a single experiment through quantification by mass spectrometry of unique peptide barcodes added to the nanobodies.

Accordingly, a monoclonal nanobody that binds specifically to a target antigen can be more efficiently manufactured at lower cost in a shorter period of time. The present invention is useful to manufacture laboratory reagents for basic imaging study and sensing study, and diagnostic agents and drugs in which a molecular target antibody is used.

### EXAMPLES

### Example 1: Production of Nanobody Fused with Peptide Barcode

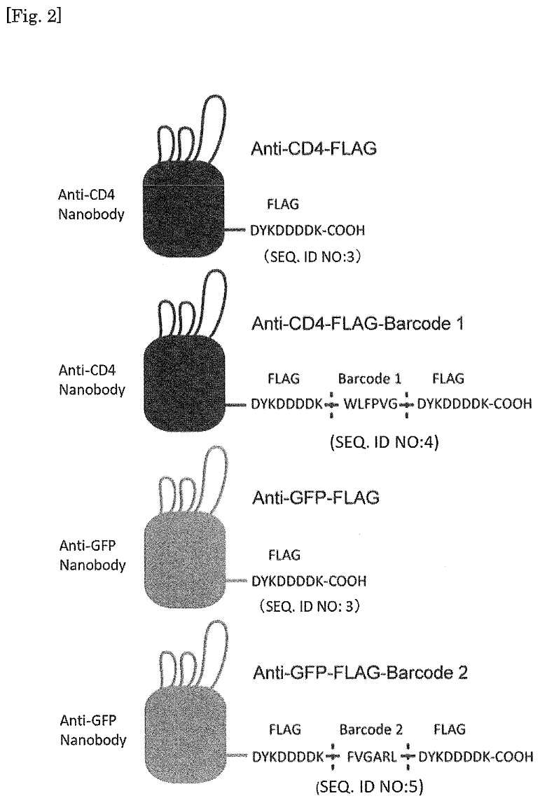

Four nanobodies were designed based on an anti-CD4 nanobody (U.S. Patent Application Publication No. 2011/0318347) and an anti-GFP (green fluorescent protein) nanobody (Mol. Cell. Proteomics. 2008:7:282-289). FIG. 2 shows schematic views of four types of nanobodies designed in this example. Two of the nanobodies were nanobodies with a unique peptide barcode having six amino acid residues, and the other two nanobodies were nanobodies with no peptide barcode. A barcode 1 was added to the anti-CD4 nanobody as the peptide barcode, and a barcode 2 was added to the anti-GFP nanobody as the peptide barcode (FIG. 2).

The peptide barcodes were designed as follows. The ionization efficiency of a peptide significantly varies depending on the length of the peptide and the constituent amino acid residues. A peptide having 6 to 16 residues exhibits a high ionization efficiency. The ionization efficiency also depends on the types of amino acid residues included in a peptide. For example, residues having an amino group, such as lysine (K) and arginine (R), are likely to be positively charged, and improve the ionization efficiency. A glycine (G) residue and a proline (P) residue also improve the ionization efficiency, and histidine (H) reduces the ionization efficiency. Cysteine (C) and methionine (M) are likely to be oxidized, and asparagine (N), serine (S), and threonine (T) can be glycosylated, and therefore, these residues are not suitable for SRM analysis, through which a predetermined target is analyzed. In addition, hydrophobic amino acids were included in order to adequately vary the elution time (retention time) in a liquid chromatograph.

In consideration of these factors, nine amino acids (A, F, G, K, L, P, R, V, and W) were selected as constituent amino acid residues, and two peptide barcodes having different molecular weights were designed.

(SEQ. ID NO: 1)
Barcode 1: WLFPVG
(SEQ. ID NO: 2)
Barcode 2: FVGARL

FLAG tag peptides (DYKDDDDK: SEQ. ID NO: 3) were fused to the N-termini of the barcodes 1 and 2. FLAG tag peptides were also fused to the C-termini of the barcodes 1 and 2 (FIG. 2).

The “FLAG tag peptide-barcode 1-FLAG tag peptide” (whose amino acid sequence is represented by SEQ. ID NO: 4) was fused to the C-terminus of the anti-CD4 nanobody, and the “FLAG tag peptide-barcode 2-FLAG tag peptide” (whose amino acid sequence is represented by SEQ. ID NO: 5) was fused to the C-terminus of the anti-GFP nanobody (these nanobodies were respectively referred to as “anti-CD4-FLAG-barcode 1” and “anti-GFP-FLAG-barcode 2”: FIG. 2).

In the case where the anti-CD4 nanobody with no barcode and the anti-GFP nanobody with no barcode, FLAG tag peptides (DYKDDDDK: SEQ. ID NO: 3) were fused to the C-termini of these nanobodies (these nanobodies were respectively referred to as “anti-CD4-FLAG” and “anti-GFP-FLAG”: FIG. 2).

Plasmids encoding these nanobodies were constructed. The plasmids were designed such that a gene encoding the α-factor secretory signal and a gene encoding a nanobody were arranged downstream of the AOX1 promoter. A DNA fragment was obtained by synthesizing a gene encoding the nanobody designed in this example (anti-CD4-FLAG, anti-CD4-FLAG-barcode 1, anti-GFP-FLAG, or anti-GFP-FLAG-barcode 2), and In Fusion HD Cloning Kit (manufactured by Takara Bio Inc.) was used to insert this DNA fragment into the pPIC9K vector (manufactured by Invitrogrn: this vector includes the AOX1 promoter and the α-factor secretory signal) that had been cleaved in advance using EcoRI and NotI (both enzymes were manufactured by TOYOBO Co., Ltd.). 30 [0104] The entire sequences of the plasmids were represented by SEQ. ID NOs: 6 to 17 (anti-CD4-FLAG plasmid: the entire sequence (SEQ. ID NO:6) and expressed peptide sequences (SEQ. ID NOs: 7 and 8); anti-CD4-FLAG-barcode 1 plasmid: the entire sequence (SEQ. ID NO: 9) and expressed peptide sequences (SEQ. ID NOs: 10 and 11); anti-GFP-FLAG plasmid: the entire sequence (SEQ. ID NO: 12) and expressed peptide sequences (SEQ. ID NOs: 13 and 14); and anti-GFP-FLAG-barcode 2 plasmid: the entire sequence (SEQ. ID NO: 15) and expressed peptide sequences (SEQ. ID NOs: 16 and 17)).

The constructed plasmid was digested using SacI (manufactured by TOYOBO Co., Ltd.) and purified using the MinElute PCR purification kit (manufactured by QIAGEN). This plasmid was transformed into the Pichia pastoris GS115 strain using the Frozen EZ Yeast Transformation II Kit (manufactured by Zymo Research). The transformed cells were seeded on an MD solid medium (1.34 w/v % yeast nitrogen base (without amino acids), 2 w/v % D-glucose, and 2 w/v % agar) and colonies were obtained. The obtained colony was inoculated in 20 mL of the BMGY culture medium (1 w/v % yeast extract, 2 w/v % peptone, 1 w/v % glycerol, 0.1 M potassium phosphate buffer solution (pH 6.0), 2.68 w/v % yeast nitrogen base (without amino acids), and 400 μg/mL biotin) and cultured at 30° C. at 250 rpm for 48 hours. The grown cells were collected through centrifugation, suspended in 10 mL of the BMMY culture medium (1 w/v % yeast extract, 2 w/v % peptone, 0.1 M potassium phosphate buffer solution (pH6.0), 2.68 w/v % yeast nitrogen base (without amino acids), 400 μg/mL biotin, and 0.5 v/v % methanol), and cultured at 30° C. at 250 rpm for 24 hours. After the culture, the BMMY culture medium was centrifuged, and the supernatant was filtrated using a 0.22 μm filter. This filtrated supernatant was analyzed through SDS-PAGE, and the production of a nanobody was confirmed.

10 mL of the filtrated supernatant was centrifuged in Amicon Ultra-15 Centrifugal Filters Ultracel-3K (Merck Millipore, Burlington, Mass., USA) at 8,000 g for 60 minutes, and then 10 mL of phosphate buffered saline (PBS) was added to the Amicon Ultra-3k unit and centrifuged at 8,000 g for 60 minutes. This buffer exchange procedure was repeated twice. A concentration process was performed while buffer exchange was performed in this manner, and the resulting concentrated solution was used as a monoclonal antibody solution.

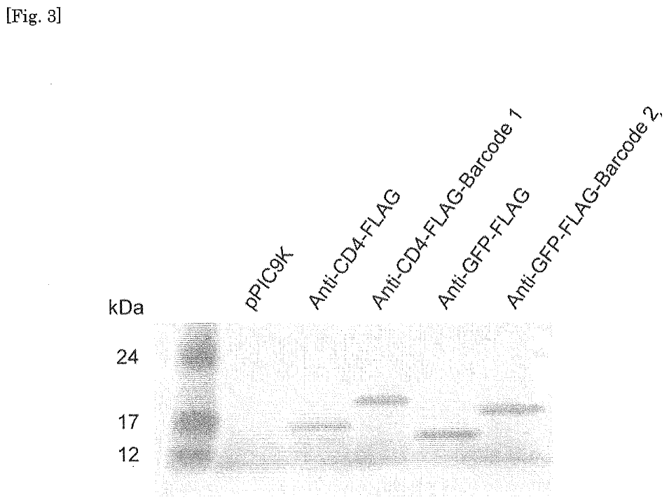

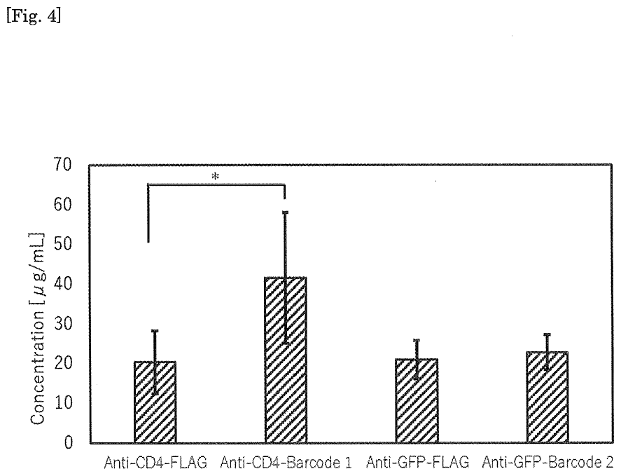

FIG. 3 is an electrophoretic photograph showing the results of SDS-PAGE of the anti-CD4-FLAG, the anti-CD4-FLAG-barcode 1, the anti-GFP-FLAG, and the anti-GFP-FLAG-barcode 2 produced by Pichia pastoris transformants. In FIG. 3, bands of the anti-CD4-FLAG, the anti-CD4-FLAG-barcode 1, the anti-GFP-FLAG, and the anti-GFP-FLAG-barcode 2 were observed at positions of the corresponding molecular weights. As is clear from the results shown in FIG. 3, it was confirmed that all the designed nanobodies were successfully produced as a result of culturing the transformants in the methanol containing culture medium. FIG. 4 is a graph showing the amounts of the anti-CD4-FLAG, the anti-CD4-FLAG-barcode 1, the anti-GFP-FLAG, and the anti-GFP-FLAG-barcode 2 produced by Pichia pastoris transformants (Each value represents mean±standard deviation obtained using at least five samples. Statistical analysis was performed based on the t-test. An asterisk represents the presence of a significant difference (p<0.05)). The productivity was slightly different between the anti-CD4-FLAG and the anti-CD4-FLAG-barcode 1 (FIG. 4). It is thought that this difference was caused by the variation in the copy number between the Pichia pastons transformants.

### Example 2: Evaluation of Binding Characteristics of Nanobody with Barcode

It was examined whether or not the addition of a peptide barcode had an influence on the nanobody characteristics.

First, surface plasmon resonance analysis was performed in accordance with the following procedure using a sensor chip on which CD4 or GFP was immobilized, and the kinetic parameters of a nanobody were measured.

The kinetic parameters were measured using Biacore T-200 (manufactured by GE Healthcare). In the case where a recombinant human sCD4 CF (manufactured by R&D Systems) and a recombinant GFP (ProSpec, Rehovot, Israel) were immobilized on Series S Sensor Chip M5 (manufactured by GE Healthcare), the analysis results therefrom were 2486.9 RU and 1189.8 RU, respectively. The anti-CD4nanobodies (the anti-CD4-FLAG and the anti-CD4-FLAG-barcode 1) were diluted using an HBS-EP buffer solution (0.01 M HEPE (pH7.4), 0.15 M NaCl, 3 mM EDTA, and 0.005 v/v % surfactant) and their concentrations were set to 0.2, 0.4, 0.6, 0.8, and 1.0 μM. The concentrations of the anti-GFP nanobodies (the anti-GFP-FLAG and the anti-GFP-FLAG-barcode 2) were set to 0.5, 1.5, 10, and 50 nM. The flow rate, the contact time, and the dissociation time were set to 30 μL/minute, 120 seconds, and 120 seconds, respectively. The CD4-immobilized chip was regenerated using 10 mM NaOH (the flow rate was 30 μL/minute and the contact time was 30 seconds), and the GFP-immobilized chip was regenerated using 50 mM NaOH (the flow rate was 30 μL/minute and the contact time was 30 seconds).

Table 1 below shows the results.

TABLE 1

| | | Kₒₙ | K_{off} | K_{D} |
|---|---|---|---|---|
| Nanobodies | Antigen | (M⁻¹s⁻¹) | (s⁻¹) | (nM) |
| Anri-CD4-FLAG | CD4 | (6.8 ± 2.3) × 10⁴ | (5.4 ± 1.5) × 10⁻³ | 42 ± 17 |
| Anti-CD4-FLAG-Barcode 1 | CD4 | (7.7 ± 0.6) × 10⁴ | (4.0 ± 0.1) × 10⁻³ | 52 ± 3 |
| Anti-GFP-FLAG | GFP | (1.1 ± 0.3) × 10⁶ | (4.8 ± 0.2) × 10⁻⁴ | 0.44 ± 0.01 |
| Anti-GFP-FLAG-Barcode 2 | GFP | (1.4 ± 0.6) × 10⁶ | (1.6 ± 1.8) × 10⁻⁴ | 0.40 ± 0.12 |

All the nanobodies bound specifically to the antigens, and there was no significant difference between the kinetic parameters of a nanobody with a peptide barcode and a nanobody with no peptide barcode (Table 1).

Next, CD4 immunofluorescence staining using a nanobody was performed. pCAGGS-CD4-myc was donated by Jacob Yount (Addgene plasmid #58537; http://n2t.net/addgene:58537; RRID:Addgene_58537). HEK293 cells were transfected with the pCAGGS-CD4-myc plasmid using Xfect Transfection Reagent (manufactured by Takara Bio Inc.) and cultured in the DMEM low-glucose culture medium (manufactured ba Nacalai Tesque Inc.) for one day. The transfected cells were moved onto a cover glass coated with poly-L-lysin hydrobromide (manufactured by Sigma-Aldrich) and culturedin the DMEMlow-glucose culture medium for 1 to 2 hours. After the culture medium had removed, the cells were fixed using 4% paraformaldehyde at room temperature for 30 minutes. The fixed cells were rinsed three times using PBS, and were blocked using PBS containing 10% FBS (manufactured by GE Healthcare) at room temperature for 30 minutes. After the anti-CD4 nanobody (anti-CD4-FLAG-barcode 1) (2.7 μg/ml) or anti-GFP nanobody (anti-GFP-FLAG-barcode 2) (3.3 μg/ml) had been incubated with the fixed cells for 90 minutes, the cells were rinsed three times using PBS and washed for 5 minutes three times using PBS. As a secondary antibody, anti-DDDDK tag mAb Alexa Fluor 488 (anti-FLAG-AF488: green under a fluorescence microscope) (manufactured by Medical & Biological Laboratories Co., Ltd.) was diluted using PBS containing 10% FBS to a concentration of 1 μg/mL, and incubated with the cells at room temperature for 1 hour. Then, the cells were rinsed three times using PBS and washed for 5 minutes three times using PBS. The nuclei were stained for 1 minute using 1 μg/mL 4′,6′-diamidino-2-phenylindoledihydrochloride (DAPI: manufactured by Nacalai Tesque Inc.) (the nuclei are stained blue). The stained cells were visualized using a confocal laser scanning fluorescence microscope LSM700 (manufactured by Carl Zeiss). It should be noted that wild-type HEK293 cells were used as a control.

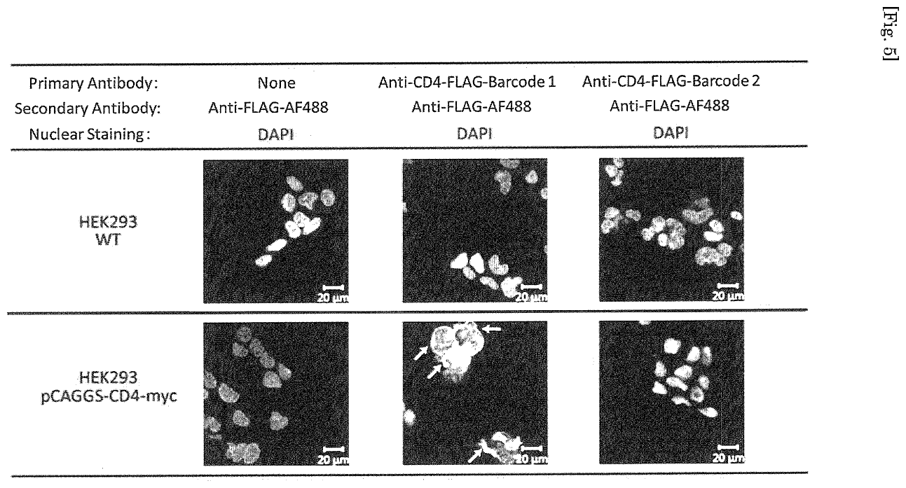

FIG. 5 shows fluorescence micrographs showing the results of CD4 immunofluorescence staining using the nanobodies with a peptide barcode. In the HEK293 cells transfected with the pCAGGS-CD4-myc (CD4 surface expressing cells) and the wild-type HEK293 cells serving as a control, staining of the nuclei was observed in all of the cells treated with the anti-CD4 nanobody (anti-CD4-FLAG-barcode 1), the cells treated with the anti-GFP nanobody (anti-GFP-FLAG-barcode 2), and the cells that were not treated with nanobodies, whereas a green color derived from the secondary antibody was observed only in the case where the anti-CD4 nanobody (anti-CD4-FLAG-barcode 1) was incubated with the CD4 surface expressing cells. Accordingly, the results shown in FIG. 5 show that the anti-CD4-FLAG-barcode 1 recognized CD4 on the surface of the HEK293 cell, but the anti-GFP-FLAG-barcode 2 did not recognize the CD4. These results show that the addition of a peptide barcode had no influence on the nanobody characteristics.

### Example 3: Characterization of Binding Ability of Nanobodies Through Mass Spectrometry

A proof-of-principle experiment for showing that a peptide barcode addition technique can be useful to measure the binding ability of a plurality of binding substances in a single experiment was designed (FIG. 6). FIG. 6 is a schematic view showing a scheme for quantifying the binding ability of nanobodies using an LC-MS. The designed experiment was as follows: first, a mixture prepared so as to contain the anti-CD4-FLAG-barcode 1 and the anti-GFP-FLAG-barcode in the same mole amount was reacted with CD4-immobilized magnetic beads (simultaneous binding assay), antibodies that did not bind in this reaction were washed away, then the peptide barcodes were cleaved from the beads by addition of enterokinase, and the cleaved peptide barcodes were quantified through a selected reaction monitoring (SRM) technique using an LC-MS or LC-MS/MS. Specifically, the experiment was performed as follows.

(Simultaneous Binding Assay: Concentration of CD4 Nanobodies Using Antigen-Antibody Interaction)

Recombinant CD4 (0.1 mg/mL) was mixed with 30 μL of NHS-activated magnetic beads (manufactured by Thermo Scientific) that had been pre-washed using 1 mM ice-cold HCl. After the reaction, the beads were washed twice using 0.1 M glycine-HCl (pH 2.0) and once using distilled water, and blocked using 3 M ethanolamine (pH 9.0) at room temperature for 2 hours. After the blocking process, the magnetic beads were suspended in 30 μL of a 50 mM borate buffer solution containing 0.05% sodium azide, and thus CD4-immobilized magnetic beads were obtained.

The CD4-immobilized magnetic beads were mixed with 500 μL of a nanobody mixture (containing 0.1 μM anti-CD4-FLAG-barcode 1 and 0.1 μM anti-GFP-FLAG-barcode 2), and then incubated at room temperature for 2 hours. After the supernatant was removed, the beads were washed twice using 1 mL of TBS containing 0.05% Tween 20, and then washed once using 1 mL of distilled water.

In order to cleave the peptide barcodes, the beads were incubated with 20 μL of 50 ng/mL enterokinase (manufactured by New England Bio-Labs) at 25° C. for 16 hours. The supernatant was desalted using MonoSpin C18 (manufactured by GL Sciences Inc.) and then lyophilized. The dried pellet was dissolved in 20 μL of 50 mM TEAB, the resulting solution was filtrated using Ultrafree-MC-HV Centrifugal Filters Durapore PVDF 0.45 μm (manufactured by Merck Millipore). The solution was stored at −20° C. until it was subjected to LC-MS/MS analysis.

(Quantification of Peptide Barcodes Using LC-MS/MS)

The peptide barcodes cleaved using enterokinase were analyzed through high performance liquid chromatography (LC) (Nexera UHPLC/HPLC system: manufactured by Shimadzu Corporation)—triple quadrupole mass spectrometry (MS/MS) (LCMS-8060: manufactured by Shimadzu Corporation).

5 μL of the solution containing the peptide barcodes was injected into 50 mm Inert SustainSwift (trade mark) C18 column (P.N. 5020-88228, inner diameter of 2.1 mm, particle diameter of 1.9 μm; manufactured by GL Sciences Inc.), to separate the barcodes. The column was kept at 40° C., and the solution was subsequently injected into the MS through a six-port injection/switching valve (manufactured by Valco Instruments). The peptide barcodes were analyzed at a flow rate of 600 μL/minute for 3.5 minutes. A gradient was formed by changing the mixing ratio of two eluents: A, 0.1 v/v % formic acid, and B, 0.1 v/v % formic acid containing acetonitrile. At the start of the gradient, the concentration of B was 5% and was kept at 5% for 0.5 minute. Then, the concentration of B was increased to 50% in 2 minutes, kept at 50% for 0.5 minutes, and increased to 95%. Lastly, the concentration of B was immediately adjusted to 5% and kept at 5% for 0.5 minutes to re-equilibrate the column. An autosampler was kept at 4° C. and provided with a black door. The temperatures of the interface, the heat block, and the desolvating unit (DL) were set to 300° C., 400° C., and 250° C., respectively. The flow rates of a nebulizer gas (N₂) for producing droplets, a drying gas (N₂), and a heating gas (dry air) were set to 2 L/minute, 10 L/minute, and 10 L/minute, respectively.

In order to optimize the SRM technique, the transitions of all the synthetic peptides (a peptide obtained by fusing a FLAG tag peptide to the C-terminus of the barcode 1 and a peptide obtained by fusing a FLAG tag peptide to the C-terminus of the barcode 2) were analyzed using Skyline software (Proteomics 2012:12:1134-1141). Two high-sensitivity transitions per peptide were selected, and collisional energy giving the highest peak intensity was employed (Table 2).

TABLE 2

| Barcode | | Precursor | Precursor | | Product | Product | Fragment |
|---|---|---|---|---|---|---|---|
| name | Peptide Sequence | ion m/z | charge | CE | ion m/z | charge | ion |
| Barcode 1 | W LFPVGDYKDDDDK | 571.60 | 3 | 20.3 | 300.17 | 1 | b 2 |
| | (SEQ. ID NO: 18) | | | | 633.78 | 2 | y 11 |
| Barcode 2 | FVGARLDYKDDDDK | 552.93 | 3 | 19.6 | 759.41 | 1 | b 7 |
| | (SEQ. ID NO: 19) | | | | 705.83 | 2 | y 12 |

Furthermore, times for these transitions were determined based on the obtained retention times. The ionized peptide barcodes were analyzed with a residence time of five minutes using this technique.

(Results)

In order to verify the sensitivity and quantitative capability of SRM, the synthetic peptides (a peptide (SEQ. ID NO: 18) obtained by fusing a FLAG tag peptide to the C-terminus of the barcode 1 and a peptide (SEQ. ID NO: 19) obtained by fusing a FLAG tag peptide to the C-terminus of the barcode 2) were serially diluted and quantified.

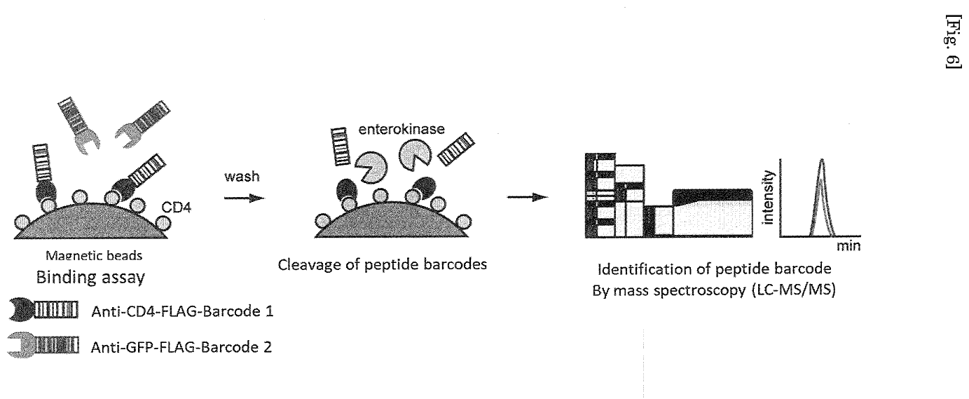

FIG. 7 shows graphs showing the LC-MS/MS analysis results of various amounts of the barcode 1 (A) and the barcode 2 (B). Each value in FIG. 7 represents mean±standard deviation obtained using three samples. It was found from the results shown in FIGS. 7A and 7B that both of the barcode 1 and the barcode 2 were detected and quantified with high sensitivity through the LC-MS/MS analysis used in this example.

As a positive control experiment, the peptide barcodes (i.e., barcode 1-FLAG (SEQ. ID NO: 18) and barcode 2-FLAG (SEQ. ID NO: 19)) were cleaved from 250 finol of the anti-CD4-FLAG-barcode 1 and 250 finol of the anti-GFP-FLAG-barcode 2 and were quantified using the LC-MS/MS.

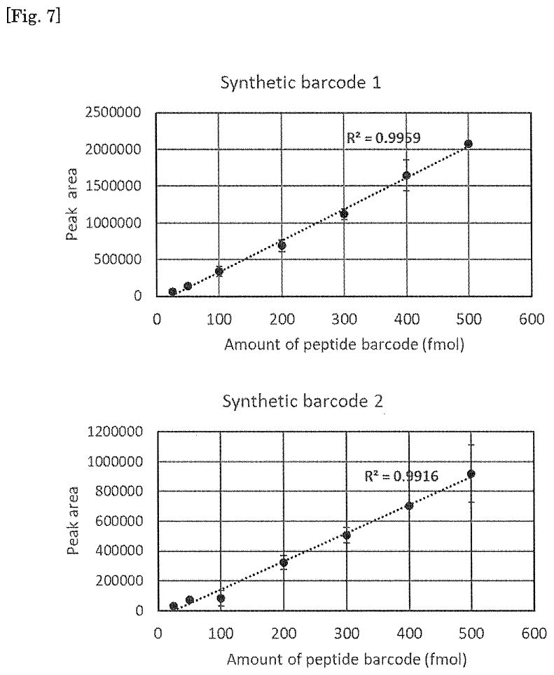

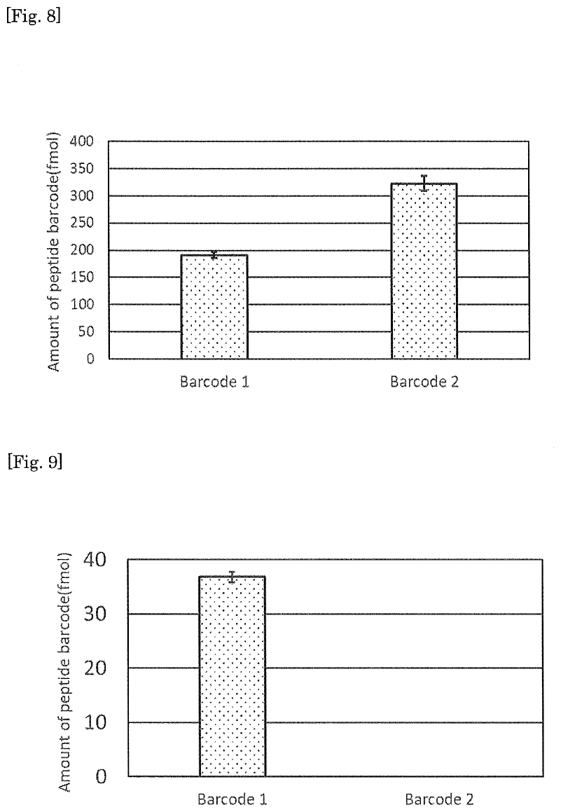

FIG. 8 is a graph showing the results of LC-MS/MS quantifications of the peptide barcodes cleaved from 250 finol of the anti-CD4-FLAG-barcode 1 and 250 finol of anti-GFP-FLAG-barcode 2. Each value in FIG. 8 represents mean±standard deviation obtained using three samples. It was found from the results shown in FIG. 8 that both of the barcode 1 and the barcode 2 were detected with high sensitivity with the LC-MS/MS used in this example and were successfully quantified with loss during the sample preparation procedure being minimized.

Next, 500 μL of a nanobody mixture (containing 0.1 μM anti-CD4-FLAG-barcode 1 and 0.1 μM anti-GFP-FLAG-barcode 2) was added to the CD4-immobilized magnetic beads to perform the simultaneous binding assay, and then the peptide barcodes cleaved from the beads were quantified.

FIG. 9 is a graph showing the results of LC-MS/MS quantifications of the peptide barcodes cleaved from the beads after the simultaneous binding assay. Each value in FIG. 9 represents mean±standard deviation obtained using three samples. As shown in FIG. 9, a peptide corresponding to the barcode 1 derived from the anti-CD4-FLAG-barcode 1 was detected, but a peptide corresponding to the barcode 2 derived from the anti-GFP-FLAG-barcode 2 was not detected. It was thus found that the peptide barcode derived from the anti-CD4-FLAG-barcode 1 that bound to a CD4 antigen held on the beads after subjected to the simultaneous binding assay was specifically detected using the LC-MS/MS.

### Example 4: Examination of Influences of Various Peptide Barcodes on Binding Assay

The following eight peptide barcodes were designed. These peptide barcodes were designed such that the degree of hydrophobicity varied therebetween. This makes it possible to adequately vary the elution time in a liquid chromatograph.

(SEQ. ID NO: 20)
Barcode 4-1: DIVVLGVEK
(SEQ. ID NO: 21)
Barcode 4-2: LIHVLDAGR
(SEQ. ID NO: 22)
Barcode 4-3: LHAILFGLPR
(SEQ. ID NO: 23)
Barcode 4-4: LEDLLLDR
(SEQ. ID NO: 24)
Barcode 4-5: LAEIHGVPR
(SEQ. ID NO: 25)
Barcode 4-6: FQFLWGPR
(SEQ. ID NO: 26)
Barcode 4-7: VELQQEVEK
(SEQ. ID NO: 27)
Barcode 4-8: NIFEQLHR

Plasmids for yeast secretory expression of the anti-CD4 nanobodies and anti-GFP nanobodies provided with the above-mentioned various peptide barcodes were constructed. The plasmids were constructed in the same manner as the construction of the plasmids of Example 1, except that the barcode moieties of the anti-CD4-FLAG-barcode 1 and the anti-GFP-FLAG-barcode 1 were changed to genes for expressing the above-mentioned various peptide barcodes. In this example, FLAG tag peptides (DYKDDDDK: SEQ. ID NO: 3) were fused to the N-termini of the peptide barcodes, but were not fused to the C-termini thereof. The base sequences of the DNA fragments encoding the fusion proteins (i.e., nanobodies with a barcode) and the expressed peptide sequences were represented by SEQ. ID NOs: 28 to 75: anti-CD4 with barcode 4-1: the base sequence of the DNA fragment (SEQ. ID NO: 28) and the expressed peptide sequences (SEQ. ID NOs: 28 to 30); anti-CD4 with barcode 4-2: the base sequence of the DNA fragment (SEQ. ID NO: 31) and the expressed peptide sequences (SEQ. ID NOs: 31 to 33); anti-CD4 with barcode 4-3: the base sequence of the DNA fragment (SEQ. ID NO: 34) and the expressed peptide sequences (SEQ. ID NOs: 34 to 36); anti-CD4 with barcode 4-4: the base sequence of the DNA fragment (SEQ. ID NO: 37) and the expressed peptide sequences (SEQ. ID NOs: 37 to 39); anti-CD4 with barcode 4-5: the base sequence of the DNA fragment (SEQ. ID NO: 40) and the expressed peptide sequences (SEQ. ID NOs: 40 to 42); anti-CD4 with barcode 4-6: the base sequence of the DNA fragment (SEQ. ID NO: 43) and the expressed peptide sequences (SEQ. ID NOs: 43 to 45); anti-CD4 with barcode 4-7: the base sequence of the DNA fragment (SEQ. ID NO: 46) and the expressed peptide sequences (SEQ. ID NOs: 46 to 48); anti-CD4 with barcode 4-8: the base sequence of the DNA fragment (SEQ. ID NO: 49) and the expressed peptide sequences (SEQ. ID NOs: 49 to 51); anti-GFP with barcode 4-1: the base sequence of the DNA fragment (SEQ. ID NO: 52) and the expressed peptide sequences (SEQ. ID NOs: 52 to 54); anti-GFP with barcode 4-2: the base sequence of the DNA fragment (SEQ. ID NO: 55) and the expressed peptide sequences (SEQ. ID NOs: 55 to 57); anti-GFP with barcode 4-3: the base sequence of the DNA fragment (SEQ. ID NO: 58) and the expressed peptide sequences (SEQ. ID NOs: 58 to 60); anti-GFP with barcode 4-4: the base sequence of the DNA fragment (SEQ. ID NO: 61) and the expressed peptide sequences (SEQ. ID NOs: 61 to 63); anti-GFP with barcode 4-5: the base sequence of the DNA fragment (SEQ. ID NO: 64) and the expressed peptide sequences (SEQ. ID NOs: 64 to 66); anti-GFP with barcode 4-6: the base sequence of the DNA fragment (SEQ. ID NO: 67) and the expressed peptide sequences (SEQ. ID NOs: 67 to 69); anti-GFP with barcode 4-7: the base sequence of the DNA fragment (SEQ. ID NO: 70) and the expressed peptide sequences (SEQ. ID NOs: 70 to 72); and anti-GFP with barcode 4-8: the base sequence of the DNA fragment (SEQ. ID NO: 73) and the expressed peptide sequences (SEQ. ID NOs: 73 to 75).

Monoclonal antibodies with a peptide barcode were obtained in the same manner as in Example 1. The ability of each of the obtained monoclonal antibodies to bind to an antigen was examined by determining kinetic parameters in the same manner as in Example 2. It should be noted that nanobodies with no peptide barcode (“anti-CD4-FLAG” and “anti-GFP-FLAG” of Example 1) were used as controls. Table 3 (CD4) and Table 4 (GFP) below shows the results. It was found that, in both cases, the addition of a peptide barcode had no influence on the ability of a nanobody to bind to an antigen.

TABLE 3

| | Kₒₙ | K_{off} | K_{D} |
|---|---|---|---|
| Barcode | (M⁻¹s⁻¹) | (s⁻¹) | (M) |
| Barcode 4-1 | 1.4 × 10⁵ | 3.0 × 10⁻³ | 2.1 × 10⁻⁸ |
| Barcode 4-2 | 1.0 × 10⁵ | 2.8 × 10⁻³ | 2.8 × 10⁻⁸ |
| Barcode 4-3 | 1.4 × 10⁵ | 3.6 × 10⁻³ | 2.6 × 10⁻⁸ |
| Barcode 4-4 | 1.2 × 10⁵ | 2.9 × 10⁻³ | 2.5 × 10⁻⁸ |
| Barcode 4-5 | 1.8 × 10⁵ | 2.8 × 10⁻³ | 2.4 × 10⁻⁸ |
| Barcode 4-6 | 1.2 × 10⁵ | 3.3 × 10⁻³ | 2.8 × 10⁻⁸ |
| Barcode 4-7 | 1.1 × 10⁵ | 2.8 × 10⁻³ | 2.4 × 10⁻⁸ |
| Barcode 4-8 | 1.6 × 10⁵ | 2.9 × 10⁻³ | 1.9 × 10⁻⁸ |
| None | 1.4 × 10⁴ | 3.7 × 10⁻³ | 2.6 × 10⁻⁸ |

TABLE 4

| | Kₒₙ | K_{off} | K_{D} |
|---|---|---|---|
| Barcode | (M⁻¹s⁻¹) | (s⁻¹) | (M) |
| Barcode 4-1 | 6.6 × 10⁵ | 3.0 × 10⁻⁴ | 4.5 × 10⁻¹⁰ |
| Barcode 4-2 | 5.6 × 10⁵ | 3.0 × 10⁻⁴ | 5.3 × 10⁻¹⁰ |
| Barcode 4-3 | 7.1 × 10⁵ | 2.1 × 10⁻⁴ | 4.3 × 10⁻¹⁰ |
| Barcode 4-4 | 6.4 × 10⁵ | 3.0 × 10⁻⁴ | 4.7 × 10⁻¹⁰ |
| Barcode 4-5 | 5.8 × 10⁵ | 3.0 × 10⁻⁴ | 5.1 × 10⁻¹⁰ |
| Barcode 4-6 | Data not shown | | |
| Barcode 4-7 | Data not shown | | |
| Barcode 4-8 | 6.0 × 10⁵ | 3.0 × 10⁻⁴ | 5.0 × 10⁻¹⁰ |
| None | 5.5 × 10⁵ | 2.6 × 10⁻⁴ | 4.6 × 10⁻¹⁰ |

### Example 5: Improvement of Detectability of Mass Spectrometer Using Long Monolith Column

In order to improve the peptide detectability of an LC-MS/MS, a long monolith column was further improved. For the purpose of evaluating the detectability, an LC-MS/MS apparatus similar to the apparatus used in Example 3 was used to measure its peak capacities. The higher the peak capacity is, the higher the separation performance is.

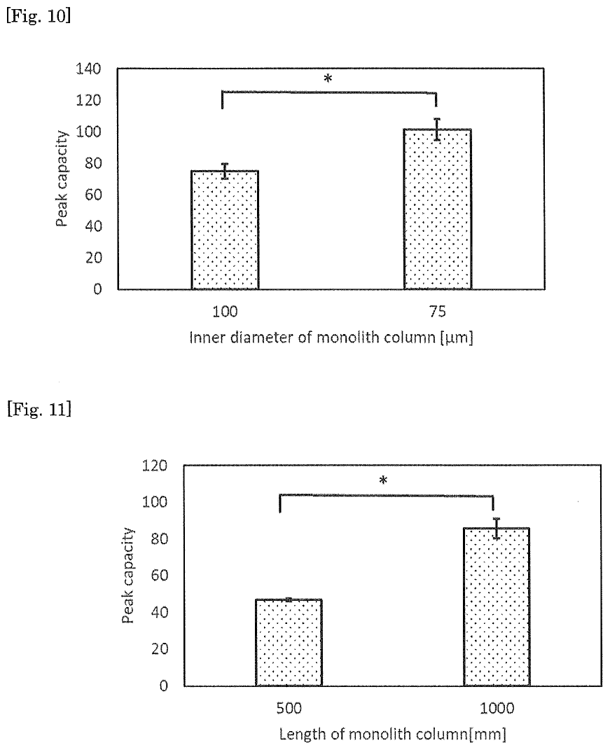

For example, FIG. 10 is a graph showing peak capacities of monolith columns with inner diameters of 100 μm and 75 μm (both of the columns had a length of 500 mm) when these columns were used for LC-MS/MS measurement of the peptide barcode 1 (SEQ. ID NO: 18). The peak capacity was improved by changing the inner diameter from 100 μm to 75 μm (FIG. 10).

Furthermore, the peak capacity was improved by increasing the length of a column. For example, FIG. 11 is a graph showing peak capacities of monolith columns with lengths of 500 mm and 1000 mm (both of the columns had an inner diameter of 75 μm) when these columns were used for LC-MS/MS measurement of the peptide barcode 1 (SEQ. ID NO: 18). It was confirmed that the peak capacity was improved by increasing the length of the column from 500 mm to 1000 mm (FIG. 11). It is expected that the performance is further improved by further increasing the length of the monolith column.

The peptide separation performance of liquid chromatography was improved by using a 1000 mm-monolith column having an inner diameter of 75 μm, and thus the peptide detection sensitivity of mass spectrometry could be significantly improved.

For example, when tryptic digests of bovine serum albumin (BSA) were analyzed through LC-MS/MS measurement using a 1000 mm-monolith column having an inner diameter of 75 μm, it was confirmed that the sensitivity was improved by a factor of two or more.

TABLE 5

| | Ratio of peak area | |
|---|---|---|
| | compared to 100 μm | |
| Peptide sequence | 100 μm | 75 μm |
| GLVLIAFSQYLQQCPFDEHVK (SEQ. ID NO: 76) | 1 ± 0.22 | 5.35 ± 0.18** |
| LVNELTEFAK (SEQ. ID NO: 77) | 1 ± 0.02 | 1.59 ± 0.16** |
| SLHTLFGDELCK (SEQ. ID NO; 78) | 1 ± 0.05 | 2.01 ± 0.13** |
| LKPDPNTLCDEFK (SEQ. ID NO: 79) | 1 ± 0.04 | 2.49 ± 0.06** |
| ECCHGDLLECADDR (SEQ. ID NO: 80) | 1 ± 0.11 | 2.12 ± 0.38* |
| DAFLGSFLYEYSR (SEQ. ID NO: 81) | 1 ± 0.07 | 1.86 ± 0.09** |
| DDPHACYSTVFDK (SEQ. ID NO: 82) | 1 ± 0.11 | 1.47 ± 0.10* |
| LGEYGFQNALIVR (SEQ. ID NO: 83) | 1 ± 0.07 | 2.74 ± 0.65* |
| RPCFSALTPDETYVPK (SEQ. ID NO: 84) | 1 ± 0.05 | 2.70 ± 0.07** |
| LFTFHADICTLPDTEK (SEQ. ID NO: 85) | 1 ± 0.05 | 2.12 ± 0.61 |
| Geometric mean | 1 | 2.28 |

(Each values represents means ± standard deviation. Underlined C represents carbamid methylated cysteine.)

### INDUSTRIAL APPLICABILITY

The present invention is useful to manufacture laboratory reagents, diagnostic agents, and drugs, for example.

1. A method for manufacturing a monoclonal antibody, comprising:
a step of introducing a DNA fragment comprising a gene that encodes a secretory signal, a gene that encodes a nanobody, and a gene that encodes a peptide barcode, or a vector containing the DNA fragment, into a yeast cell; and
a step of collecting a polypeptide comprising the nanobody and the peptide barcode that has been expressed in the cell and secreted to the outside of the cell.

2. The manufacturing method according to claim 1, wherein the yeast belongs to the genus Saccharomyces, Pichia, Schizosaccharomyces, Zygosaccharomyces, Candida, Torulopsis, Yarrowia, or Hansenula.

3. The manufacturing method according to claim 1, wherein the secretory signal is an α-factor secretory signal, a glucoamylase secretory signal, or a PHO1 secretory signal.

4. The manufacturing method according to claim 1, wherein the DNA fragment further comprises a promoter that is an AOX1 promoter, a GAP promoter, an FLD1 promoter, a PEX8 promoter, or a YPT1 promoter.

5. The manufacturing method according to claim 1, wherein the DNA fragment further comprises a gene encoding at least one tag selected from the group consisting of a FLAG tag, a His tag, a calmodulin protein (CBP) tag, a Strep tag, a StrepII tag, a GST tag, a Myc tag, and a maltose binding protein (MBP) tag.

6. The manufacturing method according to claim 1, wherein the peptide barcode is represented by an amino acid sequence having 6 to 16 amino acids, and the amino acids are independently selected from the group consisting of A, F, G, K, L, P, R, V, and W.

7. The manufacturing method according to claim 1, further comprising:
a step of mixing the collected polypeptide and an antigen and obtaining a polypeptide including a nanobody that binds specifically to the antigen;
a step of cleaving the peptide barcode from the obtained polypeptide and identifying the cleaved peptide barcode through mass spectrometry; and
a step of identifying the nanobody included in the polypeptide from which the identified peptide barcode was cleaved, based on the base sequence of a nucleic acid encoding the identified peptide barcode.

8. The manufacturing method according to claim 1, wherein the DNA fragment further comprises a specific protease cleavage site.

9. The manufacturing method according to claim 7, wherein the step of identifying the peptide barcode is performed by detecting a peak using a tandem mass spectrometer (MS/MS) connected to a high performance liquid chromatograph (LC).

10. The manufacturing method according to claim 9, wherein the high performance liquid chromatograph is provided with a long monolith column.

11. The manufacturing method according to claim 1, further comprising a step of determining the base sequence of the DNA fragment.

12. The manufacturing method according to claim 1, wherein at least two DNA fragments or vectors are used in the step of introduction into a yeast cell, and genes encoding a peptide barcode included in the DNA fragments encode peptide barcodes represented by different amino acid sequences.

13. The manufacturing method according to claim 1, wherein the DNA fragment includes a gene encoding two or more peptide barcodes, and a cleavage site is arranged at each position between the two or more peptide barcodes.

14. A vector for manufacturing a monoclonal antibody in yeast, wherein the vector contains a DNA fragment comprising a gene that encodes a secretory signal, a gene that encodes a nanobody, and a gene that encodes a peptide barcode, and is to be introduced into a cell of the yeast to express a polypeptide comprising the nanobody and the peptide barcode and secrete the polypeptide to the outside of the cell of the yeast.

15. The vector according to claim 14, wherein the secretory signal is an α-factor secretory signal, a glucoamylase secretory signal, or a PHO1 secretory signal.

16. The vector according to claim 14, wherein the DNA fragment further comprises a promoter that is an AOX1 promoter, a GAP promoter, an FLD1 promoter, a PEX8 promoter, or a YPT1 promoter.

17. The vector according to claim 14, wherein the DNA fragment further comprises a gene encoding at least one tag selected from the group consisting of a FLAG tag, a His tag, a calmodulin protein (CBP) tag, a Strep tag, a StrepII tag, a GST tag, a Myc tag, and a maltose binding protein (MBP) tag.

18. The vector according to claim 14, wherein the peptide barcode is represented by an amino acid sequence having 6 to 16 amino acids, and the amino acids are independently selected from the group consisting of A, F, G, K, L, P, R, V, and W.

19. The vector according to claim 14, wherein the DNA fragment further includes a specific protease cleavage site.

20. A screening method for a monoclonal antibody, comprising:
(i) a step of expressing an antibody library from a gene library,
the gene library comprising at least two gene members, each of the gene members comprising a DNA fragment that comprises a gene encoding a nanobody and a gene encoding at least one peptide barcode,
the DNA fragments of the gene members of the gene library encoding polypeptides of antibody members of the antibody library,
each of the polypeptides corresponding to the antibody members of the antibody library comprising a nanobody and at least one peptide barcode, the nanobody and the at least one peptide barcode being encoded by a DNA fragment comprised in a gene member of the gene library,
the peptide barcodes of the antibody members being represented by different amino acid sequences;
(ii) a step of mixing the antibody library and an antigen and selecting an antibody member of the antibody library that includes a nanobody binding to the antigen, from the antibody library;
(iii) a step of cleaving the peptide barcode included in the selected antibody member of the antibody library and identifying the cleaved peptide barcode through mass spectrometry; and
(iv) a step of determining the base sequence of the gene encoding the identified peptide barcode based on the base sequences of the gene library and identifying the nanobody of the antibody member from which the identified peptide barcode has been cleaved,
wherein the expression step is performed by introducing the vector according to claim 14 into a yeast cell.

21. The method according to claim 20, further comprising a step of determining the base sequence of the DNA fragment.


SEQUENCE LISTING (85 sequences)

SEQ ID NO: 1 - protein, 6 aa, Artificial Sequence
  Barcode 1
WLFPVG

SEQ ID NO: 2 - protein, 6 aa, Artificial Sequence
  Barcode 2
FVGARL

SEQ ID NO: 3 - protein, 8 aa, Artificial Sequence
  FLAG
DYKDDDDK

SEQ ID NO: 4 - protein, 22 aa, Artificial Sequence
  FLAG-Barcode 1-FLAG
DYKDDDDKWLFPVGDYKDDDDK

SEQ ID NO: 5 - protein, 22 aa, Artificial Sequence
  FLAG-barcode 2-FLAG
DYKDDDDKFVGARLDYKDDDDK

SEQ ID NO: 6 - DNA, 9681 bp, Artificial Sequence
  Anti-CD4-FLAG plasmid
  promoter (2)..(937): AOX1 promoter
  CDS (949)..(1638): (949)..(1215)  alpha-factor secretion signal 1 (1228)..(1602)  anti-CD4 nanobody (1612)..(1635)  FLAG
  terminator (1726)..(1972): AOX1 terminator
  rep_origin (7734)..(8416): ColE1 origin
  misc_feature (8514)..(9173): AmpR
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattcgaagttcagttagttgaatctggtggtggctctgtccaacctggtggttctcttactttgtcctgtggtacttccggtagaaccttcaacgttatgggatggttcagacaagctcctggaaaggaacgtgagtttgtcgctgctgttaggtggtcttctactggtatctattatacccaatatgctgactcagttaaaagtagatttactatttcaagagacaatgcaaagaacaccgtatacttggagatgaattcactgaagccagaggacacggctgtctactactgcgcagccgacacatataactccaatccagccagatgggacggatatgactttcgaggtcaaggaacacaggtgacagtgtccagtggaggcagtgactacaaagatgacgacgataaataagcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 7 - protein, 229 aa, Artificial Sequence
  Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAYVEFEVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGSDYKDDDDK

SEQ ID NO: 8 - protein, 229 aa, Artificial Sequence; identical to SEQ ID NO: 7
  Anti-CD4-FLAG
  MISC_FEATURE (1)..(89): alpha-factor secretion signal
  MISC_FEATURE (94)..(218): anti-CD4 nanobody
  MISC_FEATURE (222)..(229): FLAG

SEQ ID NO: 9 - DNA, 9723 bp, Artificial Sequence
  Anti-CD4-FLAG-barcode 1 plasmid
  promoter (1)..(937): AOX1 promoter
  CDS (949)..(1680): (949)..(1215) alpha-factor secretion signal (1228)..(1602)  anti-CD4 nanobody (1612)..(1635)  FLAG (1636)..(1653)  barcode 1 (1612)..(1635) FLAG
  terminator (1768)..(2014): AOX1 terminator
  rep_origin (7776)..(8458): ColE1 origin
  misc_feature (8556)..(9218): AmpR
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattcgaagttcagttagttgaatctggtggtggctctgtccaacctggtggttctcttactttgtcctgtggtacttccggtagaaccttcaacgttatgggatggttcagacaagctcctggaaaggaacgtgagtttgtcgctgctgttaggtggtcttctactggtatctattatacccaatatgctgactcagttaaaagtagatttactatttcaagagacaatgcaaagaacaccgtatacttggagatgaattcactgaagccagaggacacggctgtctactactgcgcagccgacacatataactccaatccagccagatgggacggatatgactttcgaggtcaaggaacacaggtgacagtgtccagtggaggcagtgactacaaagatgacgacgataaatggctgttccctgtcggagactataaggatgatgatgataagtaagcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 10 - protein, 243 aa, Artificial Sequence
  Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAYVEFEVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGSDYKDDDDKWLFPVGDYKDDDDK

SEQ ID NO: 11 - protein, 243 aa, Artificial Sequence; identical to SEQ ID NO: 10
  Anti-CD4-FLAG-barcode 1
  MISC_FEATURE (1)..(89): alpha-factor secretion signal
  MISC_FEATURE (94)..(218): anti-CD4 nanobody
  MISC_FEATURE (222)..(229): FLAG
  MISC_FEATURE (230)..(235): barcode 1
  MISC_FEATURE (236)..(243): FLAG

SEQ ID NO: 12 - DNA, 9654 bp, Artificial Sequence
  Anti-GFP-FLAG plasmid
  promoter (2)..(937): AOX1 promoter
  CDS (949)..(1611): (949)..(1215)  alpha-factor secretion signal 1 (1234)..(1575)  anti-GFP nanobody (1585)..(1608)  FLAG
  terminator (1699)..(1945): AOX1 terminator
  rep_origin (7707)..(8389): ColE1 origin
  misc_feature (8487)..(9146): AmpR
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattcatgcaggttcaattggttgaatctggtggtgcccttgttcaacctggaggatcactaagattatcctgtgctgcctccggatttcctgttaacaggtactccatgagatggtatagacaggctccaggtaaagaaagagagtgggtcgctggtatgtcatctgctggagatagatcctcatacgaggattctgtcaaaggaaggtttactattagtcgtgacgacgcacgtaataccgtttatttgcaaatgaattctctgaaaccagaagacactgcagtatattactgcaacgtcaacgtaggtttcgagtactggggccaaggtacacaggtgacggtgtctagtggcggtagtgactataaagatgatgatgataagtaagcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 13 - protein, 220 aa, Artificial Sequence
  Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAYVEFMQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSDYKDDDDK

SEQ ID NO: 14 - protein, 220 aa, Artificial Sequence; identical to SEQ ID NO: 13
  Anti-GFP-FLAG
  MISC_FEATURE (1)..(89): alpha-factor secretion signal
  MISC_FEATURE (96)..(209): anti-GFP nanobody
  MISC_FEATURE (213)..(220): FLAG

SEQ ID NO: 15 - DNA, 9696 bp, Artificial Sequence
  Anti-GFP-FLAG-barcode 2
  promoter (2)..(937): AOX1 promoter
  CDS (949)..(1653): (949)..(1215)  alpha-factor secretion signal 1 (1234)..(1575)  anti-GFP nanobody (1585)..(1608)  FLAG (1609)..(1626) barcode 2 (1627)..(1650) FLAG
  terminator (1741)..(1987): AOX1 terminator
  rep_origin (7749)..(8431): ColE1 origin
  misc_feature (8529)..(9188): AmpR
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaggatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagcttacgtagaattcatgcaggttcaattggttgaatctggtggtgcccttgttcaacctggaggatcactaagattatcctgtgctgcctccggatttcctgttaacaggtactccatgagatggtatagacaggctccaggtaaagaaagagagtgggtcgctggtatgtcatctgctggagatagatcctcatacgaggattctgtcaaaggaaggtttactattagtcgtgacgacgcacgtaataccgtttatttgcaaatgaattctctgaaaccagaagacactgcagtatattactgcaacgtcaacgtaggtttcgagtactggggccaaggtacacaggtgacggtgtctagtggcggtagtgactacaaagatgacgacgataaatttgtcggtgcccgtctagactacaaggatgatgatgacaagtaagcggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatctatcgaatctaaatgtaagttaaaatctctaaataattaaataagtcccagtttctccatacgaaccttaacagcattgcggtgagcatctagaccttcaacagcagccagatccatcactgcttggccaatatgtttcagtccctcaggagttacgtcttgtgaagtgatgaacttctggaaggttgcagtgttaactccgctgtattgacgggcatatccgtacgttggcaaagtgtggttggtaccggaggagtaatctccacaactctctggagagtaggcaccaacaaacacagatccagcgtgttgtacttgatcaacataagaagaagcattctcgatttgcaggatcaagtgttcaggagcgtactgattggacatttccaaagcctgctcgtaggttgcaaccgatagggttgtagagtgtgcaatacacttgcgtacaatttcaacccttggcaactgcacagcttggttgtgaacagcatcttcaattctggcaagctccttgtctgtcatatcgacagccaacagaatcacctgggaatcaataccatgttcagcttgagacagaaggtctgaggcaacgaaatctggatcagcgtatttatcagcaataactagaacttcagaaggcccagcaggcatgtcaatactacacagggctgatgtgtcattttgaaccatcatcttggcagcagtaacgaactggtttcctggaccaaatattttgtcacacttaggaacagtttctgttccgtaagccatagcagctactgcctgggcgcctcctgctagcacgatacacttagcaccaaccttgtgggcaacgtagatgacttctggggtaagggtaccatccttcttaggtggagatgcaaaaacaatttctttgcaaccagcaactttggcaggaacacccagcatcagggaagtggaaggcagaattgcggttccaccaggaatatagaggccaactttctcaataggtcttgcaaaacgagagcagactacaccagggcaagtctcaacttgcaacgtctccgttagttgagcttcatggaatttcctgacgttatctatagagagatcaatggctctcttaacgttatctggcaattgcataagttcctctgggaaaggagcttctaacacaggtgtcttcaaagcgactccatcaaacttggcagttagttctaaaagggctttgtcaccattttgacgaacattgtcgacaattggtttgactaattccataatctgttccgttttctggataggacgacgaagggcatcttcaatttcttgtgaggaggccttagaaacgtcaattttgcacaattcaatacgaccttcagaagggacttctttaggtttggattcttctttaggttgttccttggtgtatcctggcttggcatctcctttccttctagtgacctttagggacttcatatccaggtttctctccacctcgtccaacgtcacaccgtacttggcacatctaactaatgcaaaataaaataagtcagcacattcccaggctatatcttccttggatttagcttctgcaagttcatcagcttcctccctaattttagcgttcaacaaaacttcgtcgtcaaataaccgtttggtataagaaccttctggagcattgctcttacgatcccacaaggtggcttccatggctctaagaccctttgattggccaaaacaggaagtgcgttccaagtgacagaaaccaacacctgtttgttcaaccacaaatttcaagcagtctccatcacaatccaattcgatacccagcaacttttgagttgctccagatgtagcacctttataccacaaaccgtgacgacgagattggtagactccagtttgtgtccttatagcctccggaatagactttttggacgagtacaccaggcccaacgagtaattagaagagtcagccaccaaagtagtgaatagaccatcggggcggtcagtagtcaaagacgccaacaaaatttcactgacagggaactttttgacatcttcagaaagttcgtattcagtagtcaattgccgagcatcaataatggggattataccagaagcaacagtggaagtcacatctaccaactttgcggtctcagaaaaagcataaacagttctactaccgccattagtgaaacttttcaaatcgcccagtggagaagaaaaaggcacagcgatactagcattagcgggcaaggatgcaactttatcaaccagggtcctatagataaccctagcgcctgggatcatcctttggacaactctttctgccaaatctaggtccaaaatcacttcattgataccattattgtacaacttgagcaagttgtcgatcagctcctcaaattggtcctctgtaacggatgactcaacttgcacattaacttgaagctcagtcgattgagtgaacttgatcaggttgtgcagctggtcagcagcatagggaaacacggcttttcctaccaaactcaaggaattatcaaactctgcaacacttgcgtatgcaggtagcaagggaaatgtcatacttgaagtcggacagtgagtgtagtcttgagaaattctgaagccgtatttttattatcagtgagtcagtcatcaggagatcctctacgccggacgcatcgtggccgacctgcagggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccccccccctgcaggtcggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagtatctatgattggaagtatgggaatggtgatacccgcattcttcagtgtcttgaggtctcctatcagattatgcccaactaaagcaaccggaggaggagatttcatggtaaatttctctgacttttggtcatcagtagactcgaactgtgagactatctcggttatgacagcagaaatgtccttcttggagacagtaaatgaagtcccaccaataaagaaatccttgttatcaggaacaaacttcttgtttcgaactttttcggtgccttgaactataaaatgtagagtggatatgtcgggtaggaatggagcgggcaaatgcttaccttctggaccttcaagaggtatgtagggtttgtagatactgatgccaacttcagtgacaacgttgctatttcgttcaaaccattccgaatccagagaaatcaaagttgtttgtctactattgatccaagccagtgcggtcttgaaactgacaatagtgtgctcgtgttttgaggtcatctttgtatgaataaatctagtctttgatctaaataatcttgacgagccaaggcgataaatacccaaatctaaaactcttttaaaacgttaaaaggacaagtatgtctgcctgtattaaaccccaaatcagctcgtagtctgatcctcatcaacttgaggggcactatcttgttttagagaaatttgcggagatgcgatatcgagaaaaaggtacgctgattttaaacgtgaaatttatctcaagatctctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattctcatgtttgacagcttatcatcgataagctgactcatgttggtattgtgaaatagacgcagatcgggaacactgaaaaataacagttattattcg

SEQ ID NO: 16 - protein, 234 aa, Artificial Sequence
  Synthetic Construct
MRFPSIFTAVLFAASSALAAPVNTTTEDETAQIPAEAVIGYSDLEGDFDVAVLPFSNSTNNGLLFINTTIASIAAKEEGVSLEKREAEAYVEFMQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSDYKDDDDKFVGARLDYKDDDDK

SEQ ID NO: 17 - protein, 234 aa, Artificial Sequence; identical to SEQ ID NO: 16
  Anti-GFP-FLAG-barcode 2
  MISC_FEATURE (1)..(89): alpha-factor secretion signal
  MISC_FEATURE (96)..(209): anti-GFP nanobody
  MISC_FEATURE (213)..(220): FLAG
  MISC_FEATURE (221)..(226): barcode 2
  MISC_FEATURE (227)..(234): FLAG

SEQ ID NO: 18 - protein, 14 aa, Artificial Sequence
  Barcode 1-FLAG
WLFPVGDYKDDDDK

SEQ ID NO: 19 - protein, 14 aa, Artificial Sequence
  Bardose 2-FLAG
FVGARLDYKDDDDK

SEQ ID NO: 20 - protein, 9 aa, Artificial Sequence
  Barcode 4-1
DIVVLGVEK

SEQ ID NO: 21 - protein, 9 aa, Artificial Sequence
  Barcode 4-2
LIHVLDAGR

SEQ ID NO: 22 - protein, 10 aa, Artificial Sequence
  Barcode 4-3
LHAILFGLPR

SEQ ID NO: 23 - protein, 8 aa, Artificial Sequence
  Barcode4-4
LEDLLLDR

SEQ ID NO: 24 - protein, 9 aa, Artificial Sequence
  Barcode 4-5
LAEIHGVPR

SEQ ID NO: 25 - protein, 8 aa, Artificial Sequence
  Barcode 4-6
FQFLWGPR

SEQ ID NO: 26 - protein, 9 aa, Artificial Sequence
  Barcode 4-7
VELQQEVEK

SEQ ID NO: 27 - protein, 8 aa, Artificial Sequence
  Barcode 4-8
NIFEQLHR

SEQ ID NO: 28 - DNA, 438 bp, Artificial Sequence
  Anti-CD4-FLAG-barcode 4-1
  CDS (1)..(438)
gaagttcagttagttgaatctggtggtggctctgtccaacctggtggttctcttactttgtcctgtggtacttccggtagaaccttcaacgttatgggatggttcagacaagctcctggaaaggaacgtgagtttgtcgctgctgttaggtggtcttctactggtatctattatacccaatatgctgactcagttaaaagtagatttactatttcaagagacaatgcaaagaacaccgtatacttggagatgaattcactgaagccagaggacacggctgtctactactgcgcagccgacacatataactccaatccagccagatgggacggatatgactttcgaggtcaaggaacacaggtgacagtgtccagtggaggcagtgactacaaagatgacgacgataaagacatcgtcgttttaggagttgagaagtaa

SEQ ID NO: 29 - protein, 145 aa, Artificial Sequence
  Synthetic Construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGSDYKDDDDKDIVVLGVEK

SEQ ID NO: 30 - protein, 145 aa, Artificial Sequence; identical to SEQ ID NO: 29
  Anti-CD4-FLAG-barcode 4-1
  MISC_FEATURE (1)..(125): Anti-CD4
  MISC_FEATURE (129)..(136): FLAG
  MISC_FEATURE (137)..(145): Barcode 4-1

SEQ ID NO: 31 - DNA, 438 bp, Artificial Sequence
  Anti-CD4-FLAG-barcode 4-2
  CDS (1)..(438)
gaagttcagttagttgaatctggtggtggctctgtccaacctggtggttctcttactttgtcctgtggtacttccggtagaaccttcaacgttatgggatggttcagacaagctcctggaaaggaacgtgagtttgtcgctgctgttaggtggtcttctactggtatctattatacccaatatgctgactcagttaaaagtagatttactatttcaagagacaatgcaaagaacaccgtatacttggagatgaattcactgaagccagaggacacggctgtctactactgcgcagccgacacatataactccaatccagccagatgggacggatatgactttcgaggtcaaggaacacaggtgacagtgtccagtggaggcagtgactacaaagatgacgacgataaacttatccacgttcttgacgccggtagataa

SEQ ID NO: 32 - protein, 145 aa, Artificial Sequence
  Synthetic Construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGSDYKDDDDKLIHVLDAGR

SEQ ID NO: 33 - protein, 145 aa, Artificial Sequence; identical to SEQ ID NO: 32
  Anti-CD4-FLAG-barcode 4-2
  MISC_FEATURE (1)..(125): CD4
  MISC_FEATURE (129)..(136): FLAG
  MISC_FEATURE (137)..(145): Barcode 4-2

SEQ ID NO: 34 - DNA, 441 bp, Artificial Sequence
  Anti-CD4-FLAG-barcode 4-3
  CDS (1)..(441)
gaagttcagttagttgaatctggtggtggctctgtccaacctggtggttctcttactttgtcctgtggtacttccggtagaaccttcaacgttatgggatggttcagacaagctcctggaaaggaacgtgagtttgtcgctgctgttaggtggtcttctactggtatctattatacccaatatgctgactcagttaaaagtagatttactatttcaagagacaatgcaaagaacaccgtatacttggagatgaattcactgaagccagaggacacggctgtctactactgcgcagccgacacatataactccaatccagccagatgggacggatatgactttcgaggtcaaggaacacaggtgacagtgtccagtggaggcagtgactacaaagatgacgacgataaattacatgccatactatttggacttcccagataa

SEQ ID NO: 35 - protein, 146 aa, Artificial Sequence
  Synthetic Construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGSDYKDDDDKLHAILFGLPR

SEQ ID NO: 36 - protein, 146 aa, Artificial Sequence; identical to SEQ ID NO: 35
  Anti-CD4-FLAG-barcode 4-3
  MISC_FEATURE (1)..(125): CD4
  MISC_FEATURE (129)..(136): FLAG
  MISC_FEATURE (137)..(146): Barcode 4-3

SEQ ID NO: 37 - DNA, 435 bp, Artificial Sequence
  Anti-CD4-FLAG-barcode 4-4
  CDS (1)..(435)
gaagttcagttagttgaatctggtggtggctctgtccaacctggtggttctcttactttgtcctgtggtacttccggtagaaccttcaacgttatgggatggttcagacaagctcctggaaaggaacgtgagtttgtcgctgctgttaggtggtcttctactggtatctattatacccaatatgctgactcagttaaaagtagatttactatttcaagagacaatgcaaagaacaccgtatacttggagatgaattcactgaagccagaggacacggctgtctactactgcgcagccgacacatataactccaatccagccagatgggacggatatgactttcgaggtcaaggaacacaggtgacagtgtccagtggaggcagtgactacaaagatgacgacgataaattggaggatctgttgcttgatagataa

SEQ ID NO: 38 - protein, 144 aa, Artificial Sequence
  Synthetic Construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGSDYKDDDDKLEDLLLDR

SEQ ID NO: 39 - protein, 144 aa, Artificial Sequence; identical to SEQ ID NO: 38
  Anti-CD4-FLAG-barcode 4-4
  MISC_FEATURE (1)..(125): Anti-CD4
  MISC_FEATURE (129)..(136): FLAG
  MISC_FEATURE (137)..(144): Barcode 4-4

SEQ ID NO: 40 - DNA, 438 bp, Artificial Sequence
  Anti-CD4-FLAG-barcode 4-5
  CDS (1)..(438)
gaagttcagttagttgaatctggtggtggctctgtccaacctggtggttctcttactttgtcctgtggtacttccggtagaaccttcaacgttatgggatggttcagacaagctcctggaaaggaacgtgagtttgtcgctgctgttaggtggtcttctactggtatctattatacccaatatgctgactcagttaaaagtagatttactatttcaagagacaatgcaaagaacaccgtatacttggagatgaattcactgaagccagaggacacggctgtctactactgcgcagccgacacatataactccaatccagccagatgggacggatatgactttcgaggtcaaggaacacaggtgacagtgtccagtggaggcagtgactacaaagatgacgacgataaattagcagagattcatggtgttccaaggtaa

SEQ ID NO: 41 - protein, 145 aa, Artificial Sequence
  Synthetic Construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGSDYKDDDDKLAEIHGVPR

SEQ ID NO: 42 - protein, 145 aa, Artificial Sequence; identical to SEQ ID NO: 41
  Anti-CD4-FLAG-barcode 4-5
  MISC_FEATURE (1)..(125): Anti-CD4
  MISC_FEATURE (129)..(136): FLAG
  MISC_FEATURE (137)..(145): Barcode 4-5

SEQ ID NO: 43 - DNA, 435 bp, Artificial Sequence
  Anti-CD4-FLAG-barcode 4-6
  CDS (1)..(435)
gaagttcagttagttgaatctggtggtggctctgtccaacctggtggttctcttactttgtcctgtggtacttccggtagaaccttcaacgttatgggatggttcagacaagctcctggaaaggaacgtgagtttgtcgctgctgttaggtggtcttctactggtatctattatacccaatatgctgactcagttaaaagtagatttactatttcaagagacaatgcaaagaacaccgtatacttggagatgaattcactgaagccagaggacacggctgtctactactgcgcagccgacacatataactccaatccagccagatgggacggatatgactttcgaggtcaaggaacacaggtgacagtgtccagtggaggcagtgactacaaagatgacgacgataaatttcagttcttgtggggccctaggtaa

SEQ ID NO: 44 - protein, 144 aa, Artificial Sequence
  Synthetic Construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGSDYKDDDDKFQFLWGPR

SEQ ID NO: 45 - protein, 144 aa, Artificial Sequence; identical to SEQ ID NO: 44
  Anti-CD4-FLAG-barcode 4-6
  MISC_FEATURE (1)..(125): Anti-CD4
  MISC_FEATURE (129)..(136): FLAG
  MISC_FEATURE (137)..(144): Barcode 4-6

SEQ ID NO: 46 - DNA, 438 bp, Artificial Sequence
  Anti-CD4-FLAG-barcode 4-7
  CDS (1)..(438)
gaagttcagttagttgaatctggtggtggctctgtccaacctggtggttctcttactttgtcctgtggtacttccggtagaaccttcaacgttatgggatggttcagacaagctcctggaaaggaacgtgagtttgtcgctgctgttaggtggtcttctactggtatctattatacccaatatgctgactcagttaaaagtagatttactatttcaagagacaatgcaaagaacaccgtatacttggagatgaattcactgaagccagaggacacggctgtctactactgcgcagccgacacatataactccaatccagccagatgggacggatatgactttcgaggtcaaggaacacaggtgacagtgtccagtggaggcagtgactacaaagatgacgacgataaagtagaactacagcaagaagtggaaaaataa

SEQ ID NO: 47 - protein, 145 aa, Artificial Sequence
  Synthetic Construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGSDYKDDDDKVELQQEVEK

SEQ ID NO: 48 - protein, 145 aa, Artificial Sequence; identical to SEQ ID NO: 47
  Anti-CD4-FLAG-barcode 4-7
  MISC_FEATURE (1)..(125): Anti-CD4
  MISC_FEATURE (129)..(136): FLAG
  MISC_FEATURE (137)..(145): Barcode 4-7

SEQ ID NO: 49 - DNA, 435 bp, Artificial Sequence
  Anti-CD4-FLAG-barcode 4-8
  CDS (1)..(435)
gaagttcagttagttgaatctggtggtggctctgtccaacctggtggttctcttactttgtcctgtggtacttccggtagaaccttcaacgttatgggatggttcagacaagctcctggaaaggaacgtgagtttgtcgctgctgttaggtggtcttctactggtatctattatacccaatatgctgactcagttaaaagtagatttactatttcaagagacaatgcaaagaacaccgtatacttggagatgaattcactgaagccagaggacacggctgtctactactgcgcagccgacacatataactccaatccagccagatgggacggatatgactttcgaggtcaaggaacacaggtgacagtgtccagtggaggcagtgactacaaagatgacgacgataaaaatatattcgagcagcttcaccgttaa

SEQ ID NO: 50 - protein, 144 aa, Artificial Sequence
  Synthetic Construct
EVQLVESGGGSVQPGGSLTLSCGTSGRTFNVMGWFRQAPGKEREFVAAVRWSSTGIYYTQYADSVKSRFTISRDNAKNTVYLEMNSLKPEDTAVYYCAADTYNSNPARWDGYDFRGQGTQVTVSSGGSDYKDDDDKNIFEQLHR

SEQ ID NO: 51 - protein, 144 aa, Artificial Sequence; identical to SEQ ID NO: 50
  Anti-CD4-FLAG-barcode 4-8
  MISC_FEATURE (1)..(125): Anti-CD4
  MISC_FEATURE (129)..(136): FLAG
  MISC_FEATURE (137)..(144): Barcode 4-8

SEQ ID NO: 52 - DNA, 405 bp, Artificial Sequence
  Anti-GFP-FLAG-barcode 4-1
  CDS (1)..(405)
gttcaattggttgaatctggtggtgcccttgttcaacctggaggatcactaagattatcctgtgctgcctccggatttcctgttaacaggtactccatgagatggtatagacaggctccaggtaaagaaagagagtgggtcgctggtatgtcatctgctggagatagatcctcatacgaggattctgtcaaaggaaggtttactattagtcgtgacgacgcacgtaataccgtttatttgcaaatgaattctctgaaaccagaagacactgcagtatattactgcaacgtcaacgtaggtttcgagtactggggccaaggtacacaggtgacggtgtctagtggcggtagtgactataaagatgatgatgataaggacatcgtcgttttaggagttgagaagtaa

SEQ ID NO: 53 - protein, 134 aa, Artificial Sequence
  Synthetic Construct
VQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSDYKDDDDKDIVVLGVEK

SEQ ID NO: 54 - protein, 134 aa, Artificial Sequence; identical to SEQ ID NO: 53
  Anti-GFP-FLAG-barcode 4-1
  MISC_FEATURE (1)..(114): Anti-GFP
  MISC_FEATURE (118)..(125): FLAG
  MISC_FEATURE (126)..(134): Barcode 4-1

SEQ ID NO: 55 - DNA, 405 bp, Artificial Sequence
  Anti-GFP-FLAG-barcode 4-2
  CDS (1)..(405)
gttcaattggttgaatctggtggtgcccttgttcaacctggaggatcactaagattatcctgtgctgcctccggatttcctgttaacaggtactccatgagatggtatagacaggctccaggtaaagaaagagagtgggtcgctggtatgtcatctgctggagatagatcctcatacgaggattctgtcaaaggaaggtttactattagtcgtgacgacgcacgtaataccgtttatttgcaaatgaattctctgaaaccagaagacactgcagtatattactgcaacgtcaacgtaggtttcgagtactggggccaaggtacacaggtgacggtgtctagtggcggtagtgactataaagatgatgatgataagcttatccacgttcttgacgccggtagataa

SEQ ID NO: 56 - protein, 134 aa, Artificial Sequence
  Synthetic Construct
VQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSDYKDDDDKLIHVLDAGR

SEQ ID NO: 57 - protein, 134 aa, Artificial Sequence; identical to SEQ ID NO: 56
  Anti-GFP-FLAG-barcode 4-2
  MISC_FEATURE (1)..(114): Anti-GFP
  MISC_FEATURE (118)..(125): FLAG
  MISC_FEATURE (126)..(134): Barcode 4-2

SEQ ID NO: 58 - DNA, 408 bp, Artificial Sequence
  Anti-GFP-FLAG-barcode 4-3
  CDS (1)..(408)
gttcaattggttgaatctggtggtgcccttgttcaacctggaggatcactaagattatcctgtgctgcctccggatttcctgttaacaggtactccatgagatggtatagacaggctccaggtaaagaaagagagtgggtcgctggtatgtcatctgctggagatagatcctcatacgaggattctgtcaaaggaaggtttactattagtcgtgacgacgcacgtaataccgtttatttgcaaatgaattctctgaaaccagaagacactgcagtatattactgcaacgtcaacgtaggtttcgagtactggggccaaggtacacaggtgacggtgtctagtggcggtagtgactataaagatgatgatgataagttacatgccatactatttggacttcccagataa

SEQ ID NO: 59 - protein, 135 aa, Artificial Sequence
  Synthetic Construct
VQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSDYKDDDDKLHAILFGLPR

SEQ ID NO: 60 - protein, 135 aa, Artificial Sequence; identical to SEQ ID NO: 59
  Anti-GFP-FLAG-barcode 4-3
  MISC_FEATURE (1)..(114): Anti-GFP
  MISC_FEATURE (118)..(125): FLAG
  MISC_FEATURE (126)..(135): Barcode 4-3

SEQ ID NO: 61 - DNA, 402 bp, Artificial Sequence
  Anti-GFP-FLAG-barcode 4-4
  CDS (1)..(402)
gttcaattggttgaatctggtggtgcccttgttcaacctggaggatcactaagattatcctgtgctgcctccggatttcctgttaacaggtactccatgagatggtatagacaggctccaggtaaagaaagagagtgggtcgctggtatgtcatctgctggagatagatcctcatacgaggattctgtcaaaggaaggtttactattagtcgtgacgacgcacgtaataccgtttatttgcaaatgaattctctgaaaccagaagacactgcagtatattactgcaacgtcaacgtaggtttcgagtactggggccaaggtacacaggtgacggtgtctagtggcggtagtgactataaagatgatgatgataagttggaggatctgttgcttgatagataa

SEQ ID NO: 62 - protein, 133 aa, Artificial Sequence
  Synthetic Construct
VQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSDYKDDDDKLEDLLLDR

SEQ ID NO: 63 - protein, 133 aa, Artificial Sequence; identical to SEQ ID NO: 62
  Anti-GFP-FLAG-barcode 4-4
  MISC_FEATURE (1)..(114): Anti-GFP
  MISC_FEATURE (118)..(125): FLAG
  MISC_FEATURE (126)..(133): Barcode 4-4

SEQ ID NO: 64 - DNA, 405 bp, Artificial Sequence
  Anti-GFP-FLAG-barcode 4-5
  CDS (1)..(405)
gttcaattggttgaatctggtggtgcccttgttcaacctggaggatcactaagattatcctgtgctgcctccggatttcctgttaacaggtactccatgagatggtatagacaggctccaggtaaagaaagagagtgggtcgctggtatgtcatctgctggagatagatcctcatacgaggattctgtcaaaggaaggtttactattagtcgtgacgacgcacgtaataccgtttatttgcaaatgaattctctgaaaccagaagacactgcagtatattactgcaacgtcaacgtaggtttcgagtactggggccaaggtacacaggtgacggtgtctagtggcggtagtgactataaagatgatgatgataagttagcagagattcatggtgttccaaggtaa

SEQ ID NO: 65 - protein, 134 aa, Artificial Sequence
  Synthetic Construct
VQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSDYKDDDDKLAEIHGVPR

SEQ ID NO: 66 - protein, 134 aa, Artificial Sequence; identical to SEQ ID NO: 65
  Anti-GFP-FLAG-barcode 4-5
  MISC_FEATURE (1)..(114): Anti-GFP
  MISC_FEATURE (118)..(125): FLAG
  MISC_FEATURE (126)..(134): Barcode 4-5

SEQ ID NO: 67 - DNA, 402 bp, Artificial Sequence
  Anti-GFP-FLAG-barcode 4-6
  CDS (1)..(402)
gttcaattggttgaatctggtggtgcccttgttcaacctggaggatcactaagattatcctgtgctgcctccggatttcctgttaacaggtactccatgagatggtatagacaggctccaggtaaagaaagagagtgggtcgctggtatgtcatctgctggagatagatcctcatacgaggattctgtcaaaggaaggtttactattagtcgtgacgacgcacgtaataccgtttatttgcaaatgaattctctgaaaccagaagacactgcagtatattactgcaacgtcaacgtaggtttcgagtactggggccaaggtacacaggtgacggtgtctagtggcggtagtgactataaagatgatgatgataagtttcagttcttgtggggccctaggtaa

SEQ ID NO: 68 - protein, 133 aa, Artificial Sequence
  Synthetic Construct
VQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSDYKDDDDKFQFLWGPR

SEQ ID NO: 69 - protein, 133 aa, Artificial Sequence; identical to SEQ ID NO: 68
  Anti-GFP-FLAG-barcode 4-6
  MISC_FEATURE (1)..(114): Anti-GFP
  MISC_FEATURE (118)..(125): FLAG
  MISC_FEATURE (126)..(133): Barcode 4-6

SEQ ID NO: 70 - DNA, 405 bp, Artificial Sequence
  Anti-GFP-FLAG-barcode 4-7
  CDS (1)..(405)
gttcaattggttgaatctggtggtgcccttgttcaacctggaggatcactaagattatcctgtgctgcctccggatttcctgttaacaggtactccatgagatggtatagacaggctccaggtaaagaaagagagtgggtcgctggtatgtcatctgctggagatagatcctcatacgaggattctgtcaaaggaaggtttactattagtcgtgacgacgcacgtaataccgtttatttgcaaatgaattctctgaaaccagaagacactgcagtatattactgcaacgtcaacgtaggtttcgagtactggggccaaggtacacaggtgacggtgtctagtggcggtagtgactataaagatgatgatgataaggtagaactacagcaagaagtggaaaaataa

SEQ ID NO: 71 - protein, 134 aa, Artificial Sequence
  Synthetic Construct
VQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSDYKDDDDKVELQQEVEK

SEQ ID NO: 72 - protein, 134 aa, Artificial Sequence; identical to SEQ ID NO: 71
  Anti-GFP-FLAG-barcode 4-7
  MISC_FEATURE (1)..(114): Anti-GFP
  MISC_FEATURE (118)..(125): FLAG
  MISC_FEATURE (126)..(134): Barcode 4-7

SEQ ID NO: 73 - DNA, 402 bp, Artificial Sequence
  Anti-GFP-FLAG-barcode 4-8
  CDS (1)..(402)
gttcaattggttgaatctggtggtgcccttgttcaacctggaggatcactaagattatcctgtgctgcctccggatttcctgttaacaggtactccatgagatggtatagacaggctccaggtaaagaaagagagtgggtcgctggtatgtcatctgctggagatagatcctcatacgaggattctgtcaaaggaaggtttactattagtcgtgacgacgcacgtaataccgtttatttgcaaatgaattctctgaaaccagaagacactgcagtatattactgcaacgtcaacgtaggtttcgagtactggggccaaggtacacaggtgacggtgtctagtggcggtagtgactataaagatgatgatgataagaatatattcgagcagcttcaccgttaa

SEQ ID NO: 74 - protein, 133 aa, Artificial Sequence
  Synthetic Construct
VQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGSDYKDDDDKNIFEQLHR

SEQ ID NO: 75 - protein, 133 aa, Artificial Sequence; identical to SEQ ID NO: 74
  Anti-GFP-FLAG-barcode 4-8
  MISC_FEATURE (1)..(114): Anti-GFP
  MISC_FEATURE (118)..(125): FLAG
  MISC_FEATURE (126)..(133): Barcode 4-8

SEQ ID NO: 76 - protein, 21 aa, Artificial Sequence
  Peptide 1
GLVLIAFSQYLQQCPFDEHVK

SEQ ID NO: 77 - protein, 10 aa, Artificial Sequence
  Peptide 2
LVNELTEFAK

SEQ ID NO: 78 - protein, 12 aa, Artificial Sequence
  Peptide 3
SLHTLFGDELCK

SEQ ID NO: 79 - protein, 13 aa, Artificial Sequence
  Peptide 4
LKPDPNTLCDEFK

SEQ ID NO: 80 - protein, 14 aa, Artificial Sequence
  Peptide 5
ECCHGDLLECADDR

SEQ ID NO: 81 - protein, 13 aa, Artificial Sequence
  Peptide 6
DAFLGSFLYEYSR

SEQ ID NO: 82 - protein, 13 aa, Artificial Sequence
  Peptide 7
DDPHACYSTVFDK

SEQ ID NO: 83 - protein, 13 aa, Artificial Sequence
  Peptide 8
LGEYGFQNALIVR

SEQ ID NO: 84 - protein, 16 aa, Artificial Sequence
  Peptide 9
RPCFSALTPDETYVPK

SEQ ID NO: 85 - protein, 16 aa, Artificial Sequence
  Peptide 10
LFTFHADICTLPDTEK